(19) United States
(12) Patent Application Publication
(10) Pub. No.: US 2021/0047696 A1
(43) Pub. Date: Feb. 18, 2021

(54) METHODS AND PHARMACEUTICAL COMPOSITIONS FOR TREATING CANCER

(71) Applicants: INSERM (Institut National de la Santé et de la Recherche Médicale), Paris (FR); Sorbonne Université, Paris (FR); Ligue Nationale Contre le Cancer, Paris (FR)

(72) Inventors: Alex DUVAL, Paris (FR); Vincent JONCHERE, Paris (FR); Ada COLLURA, Paris (FR); Laetitia MARISA, Paris (FR); Aurélien DE REYNIES, Paris (FR)

(21) Appl. No.: 17/041,874

(22) PCT Filed: Mar. 27, 2019
(86) PCT No.: PCT/EP2019/057674

§ 371 Date: Sep. 25, 2020

(30) Foreign Application Priority Data
Mar. 28, 2018 (EP) 18305343.8

(51) Int. Cl. C12Q 1/6886; A61P 35/00; A61K 31/7105

(52) U.S. Cl. CPC C12Q 1/6886; A61K 31/7105; A61P 35/00

(57) ABSTRACT

The invention relates to method for treating cancer. Through whole exome sequencing, the inventors analyzed 47 MSI CRC and results were confirmed in a series of 53 MSI CRC. Negatively selected coding alterations in MSI CRC were further investigated for their functional role in CRC cell lines and survival impact in a cohort of 164 MSI CRC patients. Five coding negatively selected, MSI-related mutational events were demonstrated to have deleterious effects on tumor expansion, while they were associated with worse prognosis inpatients. The inventors investigated the functional consequences of the silencing of WNK1, HMGXB4, GART, and/or PRRC2C using siRNA and/or shRNA in CRC cell lines in vitro and in vivo using xenograft models. Their inactivation in CRC cells led to deleterious effects on apoptosis, proliferation and/or cell migration. The deleterious effects were greatly enhanced when several of the targets were concomitantly silenced. The prolonged silencing of these targets led to strong inhibition of tumor growth in HCT116 (MSI) and/or SW480 (MSS) xenografts. Thus, the invention relates to a compound selected from WNK1 inhibitor, HMGXB4 inhibitor, GART inhibitor, RFC3 inhibitor and/or PRRC2C inhibitor for use in the treatment of cancer, MSS CRC and MSI CRC.

### FIELD OF THE INVENTION:

The present invention relates to methods and pharmaceutical compositions for treating cancer in a subject in need thereof.

### BACKGROUND OF THE INVENTION:

Acquisition of the multiple hallmarks of cancer is mainly due to somatic mutations. These hallmarks are a convenient organizing principle to rationalize the growth and complexity of tumors (1). Underlying these mutations is the characteristic of genomic instability. This leads to the generation of mutant genotypes that confer advantages or disadvantages to the cells in which they occur, thus allowing the cells to dominate or to involute within the tumor mass (2). Data obtained from the analysis of thousands of tumors from different primary sites show that unlike species evolution, positive selection outweighed the negative selection of somatic mutational events during tumor progression (3, 4).

Different types of genomic instabilities have been described in human malignancies, including a subset of cancers that is characterized by inactivating alterations of mismatch repair (MMR) genes (5-8). These tumors exhibit a distinctive phenotype referred to as microsatellite instability (MSI), also known as the mutator phenotype, characterized by high rates of global instability at the nucleotide level. MSI affects thousands of microsatellite DNA sequences, while numerous alterations also occur in non-repetitive DNA sequences during tumor progression. This phenotype was first observed in tumors from individuals with the familial cancer condition known as Lynch syndrome, and later in sporadic colon, gastric, endometrial and other cancer types (9-12). The activating BRAF V600E somatic hotspot mutation (11), affecting a non-repetitive coding DNA sequence, plays an important role in the progression of sporadic MSI CRC. However, most somatic mutations with a postulated role in MSI tumorigenesis are found in microsatellites contained within coding regions, and to a much lesser extent in microsatellites contained within noncoding gene regions, e.g. intronic splicing areas, or in 5′ UTR or 3′ UTR (13). Because microsatellites constitute hot spots for mutations in MSI tumors regardless of their location in genes and the function of these genes, such frequent mutations could be neutral or even detrimental to tumorigenesis (14, 15). In accordance with this working hypothesis, the inventors previously reported frequent inactivation of the HSP110 oncogenic chaperone in MSI CRC (16-19).

Recent advances in high-throughput sequencing have made it possible to identify all genetic changes in human MSI neoplasms. Kim et al. reported the first global view in 27 colon and 30 endometrial tumors with the MSI phenotype (13). With regard to the selection of MSI-driven events, these authors did not take into account the strong influence of the length and nature of DNA repeats on the frequency of their instability, as demonstrated earlier by several groups (11, 15, 20). Furthermore, nucleotide instability outside of DNA microsatellites was not investigated, even though this is an important part of the landscape of somatic changes in MSI CRC (21). Other studies have attempted to identify driver genes containing selected mutations, or to use various probabilistic models of unselected mutations in MSI CRC while ignoring negative selection which is more difficult to establish (9, 22). A recent study reported that tumors with a mutator phenotype (including MMR-deficient cancers) acquired more positively selected driver mutations than other tumors, but found no evidence of negative selection (3). The latter study investigated substitutions at non-repetitive sequences, without taking into account repetitive sequences that have high physio-pathological relevance in these tumors.

In the present invention, the inventors performed whole exome sequencing of 47 MSI CRC and validated results in an independent series of 53 MSI CRC from the TCGA. In contrast to non-MSI tumors which involve mainly the positive selection of somatic mutations during their development, our results highlight the importance of both positive and negative selection of somatic mutations in MSI colon tumors. Of particular interest was the observation that (i) the majority of known target gene mutations for MSI reported previously in the literature that are thought to be key events in MSI-driven tumorigenesis was not different to the background frequency expected for their length, suggesting their overall impact on tumor development may be limited; (ii) several new mutations in small DNA repeats that showed lower frequencies but high positive selection in MSI CRC may confer strong advantages during MSI tumor progression; (iii) several negatively selected, MSI-related mutational events in coding regions have deleterious effects on tumor expansion. These rare deleterious and negatively selected alterations in MSI CRC appear to be associated with worse patient prognosis. Although requiring confirmation in independent MSI CRC cohorts, our results suggest that in the rare MSI tumors in which deleterious mutations were observed despite the negative selection, their anticancer impact should be offset by other as yet unknown oncogenic processes that contribute to poor prognosis. Overall, our findings reveal hitherto unknown pathophysiological aspects of MSI colon tumors that lead to novel therapeutic approaches specific for this tumor subtype.

### SUMMARY OF THE INVENTION:

The present invention relates to methods and pharmaceutical compositions for treating cancer in a subject in need thereof.

The present invention also relates to a compound selected from the group consisting of WNK1 inhibitor, HMGXB4 inhibitor, GART inhibitor, RFC3 inhibitor and/or PRRC2C inhibitor for use in the treatment of cancer in a subject in need thereof.

### DETAILED DESCRIPTION OF THE INVENTION

Through whole exome sequencing, 47 MSI CRC were analyzed and results were confirmed in a series of 53 MSI CRC (TCGA). The inventors used probabilistic model of mutational events within microsatellites, while adapting preexisting models to analyze non-repetitive DNA sequences. Negatively selected coding alterations in MSI CRC were further investigated for their functional role in CRC cell lines and survival impact in a cohort of 164 MSI CRC patients.

While the inventors observed the positive selection of somatic mutations in both repetitive and non-repetitive DNA sequences, negative selection of somatic mutations also frequently occurred at coding and noncoding DNA repeats in MSI CRC. Five coding negatively selected, MSI-related mutational events were demonstrated to have deleterious effects on tumor expansion, while they were associated with worse prognosis in patients.

The inventors investigated the functional consequences of the silencing of WNK1, HMGXB4, GART, RFC3 and/or PRRC2C using siRNA and/or shRNA in CRC cell lines in vitro and in vivo using xenograft models. Depending on the target gene, their inactivation in CRC cells led to deleterious effects on apoptosis, proliferation and/or cell migration. Of note, the deleterious effects were greatly enhanced when several of the targets were concomitantly silenced in the same cellular models, indicating additive effects for these events in CRC cells. In additional experiments, the prolonged silencing of these targets led to strong inhibition of tumor growth in HCT116 (MSI) and/or SW480 (MSS) xenografts.

Accordingly, the present invention relates to a compound selected from the group consisting of WNK1 inhibitor, HMGXB4 inhibitor, GART inhibitor, RFC3 inhibitor and/or PRRC2C inhibitor for use in the treatment of cancer in a subject in need thereof.

In some embodiments, the present invention relates to a compound selected from the group consisting of WNK1 inhibitor, HMGXB4 inhibitor, GART inhibitor, RFC3 inhibitor and/or PRRC2C inhibitor for use in the treatment of microsatellite stable cancer (MSS cancer) and microsatellite unstable cancer (MSI cancer) in a subject in need thereof.

In some embodiments, the present invention relates to a compound selected from the group consisting of WNK1 inhibitor, HMGXB4 inhibitor, GART inhibitor, RFC3 inhibitor and/or PRRC2C inhibitor for use in the treatment of colorectal cancer in a subject in need thereof.

Typically, 1, 2, 3, 4 or 5 compound selected from the group consisting of WNK1 inhibitor, HMGXB4 inhibitor, GART inhibitor, RFC3 inhibitor and PRRC2C inhibitor is used according to the invention.

As used herein, the term “subject” denotes a mammal. Typically, a subject according to the invention refers to any subject (preferably human) afflicted or at risk to be afflicted with cancer. Typically, a subject according to the invention refers to any subject (preferably human) afflicted or at risk to be afflicted with microsatellite stable cancer (MSS cancer) or microsatellite unstable cancer (MSI cancer). Typically, a subject according to the invention refers to any subject (preferably human) afflicted or at risk to be afflicted with colorectal cancer (CRC). In a particular embodiment, the term “subject” refers to a subject afflicted colorectal cancer receiving anti-cancer therapy. In a particular embodiment, the term “subject” refers to a subject afflicted or at risk to be afflicted with microsatellite stable colorectal cancer. In a particular embodiment, the term “subject” refers to a subject afflicted or at risk to be afflicted with microsatellite unstable colorectal cancer.

In some embodiments, the subject suffers from a microsatellite stable cancer (MSS cancer).

In some embodiments, the subject suffers from a microsatellite unstable cancer (MSI cancer).

In some embodiments, the subject suffers from a microsatellite stable colorectal cancer (MSS CRC).

In some embodiments, the subject suffers from a microsatellite unstable colorectal cancer (MSI CRC).

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may be treated by methods and compositions of the present invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxo sarcoma; liposarcoma; leiomyo sarcoma; rhabdomyo sarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the subject suffers from a cancer selected from the group consisting of colon cancer, rectal cancer, pancreatic cancer, breast cancer, lung cancer, prostate cancer, testicular cancer, brain cancer, skin cancer, gastric cancer, esophageal cancer, sarcomas, tracheal cancer, head and neck cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers.

In some embodiments, the subject suffers from cancer resistant to anti-cancer treatment. As used herein, the term “microsatellite stable cancer” has its general meaning in the art and refers to cancer liable to have a MSS phenotype. “A cancer liable to have a MSS phenotype” refers to a colorectal cancer in which microsatellite instability may be absent (MSS, Microsatellite Stability). Detecting whether microsatellite instability is present may for example be performed by genotyping microsatellite markers, such as BAT25, BAT26, NR21, NR24 and NR27, e.g. as described in Buhard et al., J Clin Oncol 24 (2), 241 (2006) and in European patent application No. EP 11 305 160.1. A cancer is defined as having a MSI phenotype if instability is detected in at least 2 microsatellite markers. On the contrary, if instability is detected in one or no microsatellite marker, then said cancer has a MSS phenotype.

As used herein, the term “microsatellite unstable cancer” has its general meaning in the art and refers to cancer liable to have a MSI phenotype. “A cancer liable to have a MSI phenotype” refers to a sporadic or hereditary cancer in which microsatellite instability may be present (MSI, Microsatellite Instability) or absent (MSS, Microsatellite Stability). Detecting whether microsatellite instability is present may for example be performed by genotyping microsatellite markers, such as BAT25, BAT26, NR21, NR24 and NR27, e.g. as described in Buhard et al., J Clin Oncol 24 (2), 241 (2006) and in European patent application No. EP 11 305 160.1. A cancer is defined as having a MSI phenotype if instability is detected in at least 2 microsatellite markers. On the contrary, if instability is detected in one or no microsatellite marker, then said cancer has a MSS phenotype. A sporadic cancer liable to have a MSI phenotype may refer to a cancer due to somatic genetic alteration of one of the Mismatch Repair (MMR) genes MLH1, MSH2, MSH6 and PMS2. For example, a sporadic cancer liable to have a MSI phenotype can be a cancer due to de novo bi-allelic methylation of the promoter of MLH1 gene. An hereditary cancer liable to have a MSI phenotype may refer to a cancer that occurs in the context of Lynch syndrome or Constitutional Mismatch-Repair Deficiency (CMMR-D). A patient suffering from Lynch syndrome is defined as a patient with an autosomal mutation in one of the 4 genes MLH1, MSH2, MSH6, and PMS2. A patient suffering from CMMR-D is defined as a patient with a germline biallelic mutation in one of the 4 genes MLH1, MSH2, MSH6, and PMS2. The MSI phenotype is present across different cancer types such as described in Ronald J Hause et al., Nat. Med 2016 (39). Accordingly, the term “microsatellite unstable cancer” refers to any cancer type having MSI phenotype. Examples of cancers liable to have a MSI phenotype include adenoma or primary tumors, such as colorectal cancer (also called colon cancer or large bowel cancer), colon adenocarcinoma, rectal adenocarcinoma, gastric cancer, stomach cancer, endometrial cancer, uterine cancer, uterine corpus endometrial carcinoma, breast cancer, bladder cancer, hepatobiliary tract cancer, liver hepatocellular carcinoma, urinary tract cancer, urothelial carcinoma, ovary cancer, ovarian serous cystadenocarcinoma, lung adenocarcinoma, lung squamous cell carcinoma, bladder cancer, prostate cancer, kidney cancer, kidney renal papillary cell carcinoma, head and neck cancer, skin cancer, skin cutaneous melanoma, thyroid carcinoma, squamous cell carcinoma, lymphomas, leukemia, brain cancer, brain lower grade glioma, glioblastoma, glioblastoma multiforme, astrocytoma, neuroblastoma and cancers described in Ronald J Hause et al., Nat. Med 2016 (39).

As used herein, the term “colorectal cancer” or “CRC” includes the well-accepted medical definition that defines colorectal cancer as a medical condition characterized by cancer of cells of the intestinal tract below the small intestine (i.e., the large intestine (colon), including the cecum, ascending colon, transverse colon, descending colon, sigmoid colon, and rectum). Additionally, as used herein, the term “colorectal cancer” also further includes medical conditions, which are characterized by cancer of cells of the duodenum and small intestine (jejunum and ileum).

The term “anti-cancer therapy” has its general meaning in the art and refers to anti-cancer compounds used in anti-cancer therapy such as tyrosine kinase inhibitors, tyrosine kinase receptor (TKR) inhibitors, EGFR tyrosine kinase inhibitors, anti-EGFR compounds, anti-HER2 compounds, Vascular Endothelial Growth Factor Receptors (VEGFRs) pathway inhibitors, interferon therapy, alkylating agents, anti-metabolites, immunotherapeutic agents, Interferons (IFNs), Interleukins, and chemotherapeutic agents such as described below.

The term “tyrosine kinase inhibitor” or “TKI” has its general meaning in the art and refers to any of a variety of therapeutic agents or drugs such as compounds inhibiting tyrosine kinase, tyrosine kinase receptor inhibitors (TKRI), EGFR tyrosine kinase inhibitors, EGFR antagonists. The term “tyrosine kinase inhibitor” or “TKI” has its general meaning in the art and refers to any of a variety of therapeutic agents or drugs that act as selective or non-selective inhibitors of receptor and/or non-receptor tyrosine kinases. Tyrosine kinase inhibitors and related compounds are well known in the art and described in U.S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to Erlotinib, sunitinib (Sutent; SU11248), dasatinib (BMS-354825), PP2, BEZ235, saracatinib, gefitinib (Iressa), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo[3,4-f][1,6]naphthyridin-3(2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In certain embodiments, the tyrosine kinase inhibitor is a small molecule kinase inhibitor that has been orally administered and that has been the subject of at least one Phase I clinical trial, more preferably at least one Phase II clinical, even more preferably at least one

Phase III clinical trial, and most preferably approved by the FDA for at least one hematological or oncological indication. Examples of such inhibitors include, but are not limited to Erlotinib, Gefitinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

EGFR tyrosine kinase inhibitors as used herein include, but are not limited to compounds selected from the group consisting of but not limited to Erlotinib, lapatinib, Rociletinib (CO-1686), gefitinib, Dacomitinib (PF-00299804), Afatanib, Brigatinib (AP26113), WJTOG3405, NEJ002, AZD9291, HM61713, EGF816, ASP 8273, AC 0010. Examples of antibody EGFR inhibitors include Cetuximab, panitumumab, matuzumab, zalutumumab, nimotuzumab, necitumumab, Imgatuzumab (GA201, R05083945), and ABT-806.

The term “WNK1” has its general meaning in the art and refers to Lysine Deficient Protein Kinase 1, a member of the WNK subfamily of serine/threonine protein kinases which codes for a positive regulator of canonical Wnt/-catenin signaling (41, 42).

The term “HMGXB4” has its general meaning in the art and refers to HMG-Box Containing 4, which codes for a histone chromosomal protein belonging to the High Mobility Group (HMG)-box protein family.

The term “GART” has its general meaning in the art and refers to Phosphoribosylglycinamide Formyltransferase (PGFT) and Phosphoribosylglycinamide Synthetase (PRGS), which codes for a phospho-ribosylglycinamide formyltransferase (43, 44).

The term “RFC3” has its general meaning in the art and refers to Replication Factor C Subunit 3 (45, 46).

The term “PRRC2C” has its general meaning in the art and refers to Proline Rich Coiled-Coil 2C protein (47).

In some embodiments, the present invention relates to a WNK1 inhibitor for use according to the invention.

In some embodiments, the present invention relates to a HMGXB4 inhibitor for use according to the invention. In some embodiments, the present invention relates to a GART inhibitor for use according to the invention.

In some embodiments, the present invention relates to a RFC3 inhibitor for use according to the invention.

In some embodiments, the present invention relates to a PRRC2C inhibitor for use according to the invention.

In some embodiments, the present invention relates to WNK1 inhibitor and HMGXB4 inhibitor for use according to the invention.

In some embodiments, the present invention relates to WNK1 inhibitor and GART inhibitor for use according to the invention.

In some embodiments, the present invention relates to WNK1 inhibitor and RFC3 inhibitor for use according to the invention.

In some embodiments, the present invention relates to WNK1 inhibitor and PRRC2C inhibitor for use according to the invention.

In some embodiments, the present invention relates to WNK1 inhibitor, HMGXB4 inhibitor and GART inhibitor for use according to the invention.

In some embodiments, the present invention relates to WNK1 inhibitor, HMGXB4 inhibitor and RFC3 inhibitor for use according to the invention.

In some embodiments, the present invention relates to WNK1 inhibitor, HMGXB4 inhibitor and PRRC2C inhibitor for use according to the invention.

In some embodiments, the present invention relates to WNK1 inhibitor, GART inhibitor and RFC3 inhibitor for use according to the invention.

In some embodiments, the present invention relates to WNK1 inhibitor, GART inhibitor and PRRC2C inhibitor for use according to the invention.

In some embodiments, the present invention relates to WNK1 inhibitor, RFC3 inhibitor and PRRC2C inhibitor for use according to the invention.

In some embodiments, the present invention relates to WNK1 inhibitor, HMGXB4 inhibitor, GART inhibitor and RFC3 inhibitor for use according to the invention.

In some embodiments, the present invention relates to WNK1 inhibitor, HMGXB4 inhibitor, GART inhibitor and PRRC2C inhibitor for use according to the invention.

In some embodiments, the present invention relates to HMGXB4 inhibitor and GART inhibitor for use according to the invention.

In some embodiments, the present invention relates to HMGXB4 inhibitor and RFC3 inhibitor for use according to the invention.

In some embodiments, the present invention relates to HMGXB4 inhibitor and PRRC2C inhibitor for use according to the invention.

In some embodiments, the present invention relates to HMGXB4 inhibitor, GART inhibitor and RFC3 inhibitor for use according to the invention.

In some embodiments, the present invention relates to HMGXB4 inhibitor, GART inhibitor and PRRC2C inhibitor for use according to the invention.

In some embodiments, the present invention relates to HMGXB4 inhibitor, RFC3 inhibitor and PRRC2C inhibitor for use according to the invention.

In some embodiments, the present invention relates to HMGXB4 inhibitor, GART inhibitor, RFC3 inhibitor and PRRC2C inhibitor for use according to the invention.

In some embodiments, the present invention relates to GART inhibitor and RFC3 inhibitor for use according to the invention.

In some embodiments, the present invention relates to GART inhibitor and PRRC2C inhibitor for use according to the invention.

In some embodiments, the present invention relates to GART inhibitor, RFC3 inhibitor and PRRC2C inhibitor for use according to the invention.

In some embodiments, the present invention relates to RFC3 inhibitor and PRRC2C inhibitor for use according to the invention.

In some embodiments, the present invention relates to WNK1 inhibitor, HMGXB4 inhibitor, GART inhibitor, RFC3 inhibitor and PRRC2C inhibitor for use according to the invention.

In some embodiments, any combination described above is used in the treatment of colorectal cancer in a subject in need thereof.

The term “inhibitor” has its general meaning in the art and refers to a compound that selectively blocks or inactivates the target (WNK1, HMGXB4, GART, RFC3 and/or PRRC2C).

The term “inhibitor” also refers to a compound that selectively blocks the binding of the target to its substrate. The term “inhibitor” also refers to a compound able to prevent the action of the target for example by inhibiting the target controls of downstream effectors such as inhibiting the activation of the target pathway signaling. As used herein, the term “selectively blocks or inactivates” refers to a compound that preferentially binds to and blocks or inactivates the target with a greater affinity and potency, respectively, than its interaction with the other sub-types of the target family. Compounds that block or inactivate the target, but that may also block or inactivate other target sub-types, as partial or full inhibitors, are contemplated. The term “inhibitor” also refers to a compound that inhibits the target expression. Typically, an inhibitor is a small organic molecule, a polypeptide, an aptamer, an antibody, an oligonucleotide or a ribozyme.

Tests and assays for determining whether a compound is an inhibitor are well known by the skilled person in the art such as described in (41-47).

In some embodiments, WNK1 inhibitor, HMGXB4 inhibitor, GART inhibitor, RFC3 inhibitor and/or PRRC2C inhibitor is siRNA or shRNA such as described in the example.

In one embodiment of the invention, GART inhibitors include but are not limited to Pemetrexed.

In another embodiment, the target inhibitor of the invention is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against the target of the invention as above described, the skilled man in the art can easily select those blocking or inactivating the target.

In another embodiment, the target inhibitor of the invention is an antibody (the term including “antibody portion”) directed against the target.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody.

In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of the target. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the antigen may be provided as synthetic peptides corresponding to antigenic regions of interest in the target. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc' and Fc regions, for example, are effectors of the complement cascade but are not involved in antigen binding. An antibody from which the pFc' region has been enzymatically cleaved, or which has been produced without the pFc' region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc' regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgGl, IgG2, IgG3 and IgG4. In a preferred embodiment, the inhibitor of the invention is a Human IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874, 541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In one embodiment, the target inhibitor of the invention is a target expression inhibitor.

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs, which are modified, by processes such as capping, polyadenylation, methylation, and editing, and proteins modified by, for example, methylation, acetylation, phosphorylation, ubiquitination, SUMOylation, ADP-ribosylation, myristilation, and glycosylation.

An “inhibitor of expression” refers to a natural or synthetic compound that has a biological effect to inhibit the expression of a gene.

The target expression inhibitors for use in the present invention may be based on antisense oligonucleotide constructs. Anti-sense oligonucleotides, including anti-sense RNA molecules and anti-sense DNA molecules, would act to directly block the translation of the target mRNA by binding thereto and thus preventing protein translation or increasing mRNA degradation, thus decreasing the level of the target proteins, and thus activity, in a cell. For example, antisense oligonucleotides of at least about 15 bases and complementary to unique regions of the mRNA transcript sequence encoding the target can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as a target expression inhibitors for use in the present invention. The target gene expression can be reduced by contacting the subject or cell with a small double stranded RNA (dsRNA), or a vector or construct causing the production of a small double stranded RNA, such that the target expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as a target expression inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of the target mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful a target inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing the target. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol.7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

Typically the inhibitors according to the invention as described above are administered to the patient in a therapeutically effective amount.

By a “therapeutically effective amount” ofthe inhibitor ofthe present invention as above described is meant a sufficient amount of the inhibitor for treating cancer at a reasonable benefit/risk ratio applicable to any medical treatment. It will be understood, however, that the total daily usage of the inhibitors and compositions of the present invention will be decided by the attending physician within the scope of sound medical judgment. The specific therapeutically effective dose level for any particular patient will depend upon a variety of factors including the disorder being treated and the severity of the disorder; activity of the specific inhibitor employed; the specific composition employed, the age, body weight, general health, sex and diet of the patient; the time of administration, route of administration, and rate of excretion of the specific inhibitor employed; the duration of the treatment; drugs used in combination or coincidental with the specific inhibitor employed; and like factors well known in the medical arts. For example, it is well within the skill of the art to start doses of the inhibitor at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the inhibitor of the present invention for the symptomatic adjustment of the dosage to the patient to be treated. A medicament typically contains from about 0.01 mg to about 500 mg of the inhibitor of the present invention, preferably from 1 mg to about 100 mg of the inhibitor of the present invention. An effective amount of the drug is ordinarily supplied at a dosage level from 0.0002 mg/kg to about 20 mg/kg of body weight per day, especially from about 0.001 mg/kg to 7 mg/kg of body weight per day.

In a particular embodiment, the compound according to the invention may be used in a concentration between 0.01 μM and 20 μM, particularly, the compound of the invention may be used in a concentration of 0.01, 0.05, 0.1, 0.5, 1.0, 2.5, 5.0, 10.0, 15.0, 20.0 μM.

According to the invention, the compound of the present invention is administered to the subject in the form of a pharmaceutical composition. Typically, the compound of the present invention may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The compound of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized agent of the present inventions into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the compound of the present invention plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In some embodiments, the compound of the present invention is administered sequentially or concomitantly with one or more therapeutic active agent such as chemotherapeutic or radiotherapeutic.

In some embodiments, the compound of the present invention is administered with a chemotherapeutic agent. The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmo fur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; amino levulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine;

pentostatin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the compound of the present invention is administered with a targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs”, “molecularly targeted therapies”, “precision medicines”, or similar names. In some embodiments, the targeted therapy consists of administering the subject with a tyrosine kinase inhibitor as defined above.

In some embodiments, compound of the present invention is administered with an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the subject with an amount of immune cells (T cells, NK, cells, dendritic cells, B cells . . . ). Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents. Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants. A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). In addition to having specific or non-specific targets, immunotherapeutic agents can be active, i.e. stimulate the body's own immune response, or they can be passive, i.e. comprise immune system components that were generated external to the body. Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins. Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Other examples include anti-CTLA4 antibodies (e.g. Ipilimumab), anti-PD1 antibodies, anti-PDLL antibodies, anti-TIMP3 antibodies, anti-LAG3 antibodies, anti-B7H3 antibodies, anti-B7H4 antibodies or anti-B7H6 antibodies. In some embodiments, antibodies include B cell depleting antibodies. Typical B cell depleting antibodies include but are not limited to anti-CD20 monoclonal antibodies [e.g. Rituximab (Roche), Ibritumomab tiuxetan (Bayer Schering), Tositumomab (GlaxoSmithKline), AME-133v (Applied Molecular Evolution), Ocrelizumab (Roche), Ofatumumab (HuMax-CD20, Gemnab), TRU-015 (Trubion) and IMMU-106 (Immunomedics)], an anti-CD22 antibody [e.g. Epratuzumab, Leonard et al., Clinical Cancer Research (Z004) 10: 53Z7-5334], anti-CD79a antibodies, anti-CD27 antibodies, or anti-CD19 antibodies (e.g. U.S. Pat. No. 7,109,304), anti-BAFF-R antibodies (e.g. Belimumab, GlaxoSmithKline), anti-APRIL antibodies (e.g. anti-human APRIL antibody, ProSci inc.), and anti-IL-6 antibodies [e.g. previously described by De Benedetti et al., J Immunol (2001) 166: 4334-4340 and by Suzuki et al., Europ J of Immunol (1992) 22 (8) 1989-1993, fully incorporated herein by reference]. The immunotherapeutic treatment may consist of allografting, in particular, allograft with hematopoietic stem cell HSC. The immunotherapeutic treatment may also consist in an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the subject's circulating lymphocytes, NK cells, are isolated amplified in vitro and readministered to the subject. The activated lymphocytes or NK cells are most preferably be the subject's own cells that were earlier isolated from a blood or tumor sample and activated (or “expanded”) in vitro.

In some embodiments, the compound of the present invention is administered with a radiotherapeutic agent. The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

In one embodiment, said additional active compounds may be contained in the same composition or administrated separately.

In another embodiment, the pharmaceutical composition of the invention relates to combined preparation for simultaneous, separate or sequential use in the treatment of cancer in a subject in need thereof.

The invention also provides kits comprising the compound of the invention. Kits containing the compound of the invention find use in therapeutic methods.

A further aspect, the invention relates to a method of treating cancer in a subject in need thereof, comprising administering to the subject a therapeutically effective amount of a compound selected from the group consisting of WNK1 inhibitor, HMGXB4 inhibitor, GART inhibitor, RFC3 inhibitor and/or PRRC2C inhibitor.

A further aspect, the invention relates to a method of treating microsatellite stable cancer (MSS cancer) and microsatellite unstable cancer (MSI cancer) in a subject in need thereof, comprising administering to the subject a therapeutically effective amount of a compound selected from the group consisting of WNK1 inhibitor, HMGXB4 inhibitor, GART inhibitor, RFC3 inhibitor and/or PRRC2C inhibitor.

A further aspect, the invention relates to a method of treating colorectal cancer (CRC) in a subject in need thereof, comprising administering to the subject a therapeutically effective amount of a compound selected from the group consisting of WNK1 inhibitor, HMGXB4 inhibitor, GART inhibitor, RFC3 inhibitor and/or PRRC2C inhibitor.

In a further aspect, the present invention relates to a method of screening a candidate compound for use as a drug for treating cancer in a patient in need thereof, wherein the method comprises the steps of:
providing a WNK1, HMGXB4, GART, RFC3 and/or PRRC2C, providing a cell, tissue sample or organism expressing a WNK1, HMGXB4, GART, RFC3 and/or PRRC2C,
providing a candidate compound such as a small organic molecule, a polypeptide, an aptamer, an antibody or an intra-antibody,
measuring the WNK1, HMGXB4, GART, RFC3 and/or PRRC2C activity,
and selecting positively candidate compounds that inhibit WNK1, HMGXB4, GART, RFC3 and/or PRRC2C activity.

Methods for measuring WNK1, HMGXB4, GART, RFC3 and/or PRRC2C activity are well known in the art (41-47). For example, measuring the WNK1, HMGXB4, GART, RFC3 and/or PRRC2C activity involves determining a Ki on the WNK1, HMGXB4, GART, RFC3 and/or PRRC2C cloned and transfected in a stable manner into a CHO cell line, measuring cancer cell migration and invasion abilities, measuring cancer cell growth, measuring cancer cell proliferation, and measuring WNK1, HMGXB4, GART, RFC3 and/or PRRC2C pathway signaling in the present or absence of the candidate compound.

Tests and assays for screening and determining whether a candidate compound is a WNK1, HMGXB4, GART, RFC3 and/or PRRC2C inhibitor are well known in the art (41-47). In vitro and in vivo assays may be used to assess the potency and selectivity of the candidate compounds to inhibit WNK1, HMGXB4, GART, RFC3 and/or PRRC2C activity.

Activities of the candidate compounds, their ability to bind WNK1, HMGXB4, GART, RFC3 and/or PRRC2C and their ability to inhibit WNK1, HMGXB4, GART, RFC3 and/or PRRC2C activity may be tested using isolated cancer cell, cancer cell lines or CHO cell line cloned and transfected in a stable manner by the human WNK1, HMGXB4, GART, RFC3 and/or PRRC2C.

A further object of the invention relates to a method for predicting the survival time of a subject suffering from a cancer comprising i) detecting at least one mutation of WNK1, HMGXB4, GART, RFC3 and/or PRRC2C in a biological sample obtained from the subject, and ii) concluding that the subject will have a short survival time when detecting said at least one mutation of WNK1, HMGXB4, GART, RFC3 and/or PRRC2C.

A further object of the invention relates to a method for predicting the survival time of a subject suffering from a microsatellite stable cancer (MSS cancer) or a microsatellite unstable cancer (MSI cancer) comprising i) detecting at least one mutation of WNK1, HMGXB4, GART, RFC3 and/or PRRC2C in a biological sample obtained from the subject, and ii) concluding that the subject will have a short survival time when detecting said at least one mutation of WNK1, HMGXB4, GART, RFC3 and/or PRRC2C.

A further object of the invention relates to a method for predicting the survival time of a subject suffering from a colorectal cancer (CRC) comprising i) detecting at least one mutation of WNK1, HMGXB4, GART, RFC3 and/or PRRC2C in a biological sample obtained from the subject, and ii) concluding that the subject will have a short survival time when detecting said at least one mutation of WNK1, HMGXB4, GART, RFC3 and/or PRRC2C.

The term “mutation” has its general meaning in the art and refers to a coding mutations affecting WNK1, HMGXB4, GART, RFC3 and/or PRRC2C. The term “mutation” of the invention also refers to mutation in the WNK1, HMGXB4, GART, RFC3 and/or PRRC2C coding region, nonsynonymous mutation, and missense mutations. The term “mutation” of the invention also refers to negatively selected coding sequence mutation. The term “mutation” of the invention also refers to mutation of microsatellites located in coding regions of WNK1, HMGXB4, PRRC2C, RFC3 and/or GART.

The step of detecting if at least one mutation of WNK1, HMGXB4, GART, RFC3 and/or PRRC2C is present in a biological sample may be performed by any method well-known by the skilled person. Particularly, the step of detecting if at least one mutation of WNK1, HMGXB4, GART, RFC3 and/or PRRC2C is present in a biological sample may be performed by the method described in the example. More particularly, said step of detecting the presence or not of at least one mutation of WNK1, HMGXB4, GART, RFC3 and/or PRRC2C may comprise:
sequencing the WNK1, HMGXB4, GART, RFC3 and/or PRRC2C gene from the DNA present in the biological sample or sequencing the WNK1, HMGXB4, GART, RFC3 and/or PRRC2C cDNA corresponding to the mRNA present in the biological sample, and
comparing the obtained sequence to a reference sequence encoding a functional WNK1, HMGXB4, GART, RFC3 and/or PRRC2C protein or comparing the amino acid sequence encoded by the obtained sequence to a reference sequence of a functional WNK1, HMGXB4, GART, RFC3 and/or PRRC2C protein.

The sequence comparison may be performed by any method well-known by the skilled person such as sequence alignment.

The method of the present invention is particularly suitable for predicting the duration of the overall survival (OS), progression-free survival (PFS) and/or the disease-free survival (DFS) of the cancer patient. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the patient will have a survival time that will be lower than the median (or mean) observed in the general population of patients suffering from said cancer. When the patient will have a short survival time, it is meant that the patient will have a “poor prognosis”. Inversely, the expression “long survival time” indicates that the patient will have a survival time that will be higher than the median (or mean) observed in the general population of patients suffering from said cancer. When the patient will have a long survival time, it is meant that the patient will have a “good prognosis”.

In some embodiments, the method of the present invention is particularly suitable for predicting relapse-free survival (RFS) of the cancer patient.

As used herein, the term “biological sample” refers to any biological sample derived from the patient such as blood sample, plasma sample, serum sample, biopsy sample, or tumor tissue sample.

As used herein, the term “tumor tissue sample” means any tissue tumor sample derived from the patient. Said tissue sample is obtained for the purpose of the in vitro evaluation. In some embodiments, the tumor sample may result from the tumor resected from the patient. In some embodiments, the tumor sample may result from a biopsy performed in the primary tumour of the patient or performed in metastatic sample distant from the primary tumor of the patient. For example an endoscopical biopsy performed in the bowel of the patient suffering from the colorectal cancer. In some embodiments, the tumor tissue sample encompasses (i) a global primary tumor (as a whole), (ii) a tissue sample from the center of the tumor, (iii) a tissue sample from the tissue directly surrounding the tumor which tissue may be more specifically named the “invasive margin” of the tumor, (iv) lymphoid islets in close proximity with the tumor, (v) the lymph nodes located at the closest proximity of the tumor, (vi) a tumor tissue sample collected prior surgery (for follow-up of patients after treatment for example), and (vii) a distant metastasis. As used herein the “invasive margin” has its general meaning in the art and refers to the cellular environment surrounding the tumor. In some embodiments, the tumor tissue sample, irrespective of whether it is derived from the center of the tumor, from the invasive margin of the tumor, or from the closest lymph nodes, encompasses pieces or slices of tissue that have been removed from the tumor center of from the invasive margin surrounding the tumor, including following a surgical tumor resection or following the collection of a tissue sample for biopsy, for further quantification of one or several biological markers, notably through histology or immunohistochemistry methods, and through methods of gene or protein expression analysis, including genomic and proteomic analysis. The tumor tissue sample can be subjected to a variety of well-known post-collection preparative and storage techniques (e.g., fixation, storage, freezing, etc.).

In some embodiments, the method of the present invention comprises detecting at least one mutation of at least one gene (i.e. 1, 2, 3, 4, or 5 genes) selected from the group consisting of WNK1, HMGXB4, GART, RFC3 and PRRC2C.

In a further aspect, the present invention relates to a method of treating a cancer in a patient identified as having a short survival time comprising the steps of:

i) determining whether the patient will have a short survival time by performing the method according to the invention, and

ii) administering a compound selected from the group consisting of anti-cancer compound, WNK1 inhibitor, HMGXB4 inhibitor, GART inhibitor, RFC3 inhibitor and/or PRRC2C inhibitor if said patient was being classified as having a short survival time.

The invention will be further illustrated by the following figures and examples. However, these examples and figures should not be interpreted in any way as limiting the scope of the present invention.

### FIGURES

FIG. 1. Validation of the deleterious effects of 5 outlier mutations on CRC cell lines

A) Flow cytometry analysis of the apoptosis (Annexin-V) of untreated (white) or TRAIL-treated (black) HCT116 (MSI, left panel) and SW480 (MSS, right panel) CRC cell lines transfected with either single specific siRNA gene (WNK1, HMGXB4, GART, RFC3 and/or PRRC2C) or with scrambled siRNA (upper panels). In the lower panel, simultaneous downregulation of three genes shows an additive effect for increasing the percentage of apoptotic cells in some cases. Data represent the mean ±SEM of three independent experiments. t-test: *p<0.05, **p<0.01, ***p<0.001, ****p<0.0001 of indicated silencing condition compared to control (si scramble in black; single siRNA indicated gene in blue). These results were confirmed in 4 other cell lines (MSI: RKO, KM12 and MSS: FET, SW620; FIG. 3). Data from FACS analysis are not shown.

B) and C) In order to evaluate the effect of silencing of the 5 genes with OM on the proliferation and migration rate of HCT116 cell lines, real-time monitoring of cell growth (B) and cell migration (C) using the xCELLigence system was performed. This system estimates the cell index in real time using a parameter based on impedance measurement and reflecting the number of cells attached to the surface of the experimental chambers. With this instrument, silencing of the WNK1, HMGXB4 and GART genes was shown to significantly attenuate cell proliferation and migration (FIG. 4). Cell index is presented as mean ±SEM of three independent experiments performed in quadruplicate. Two-way ANOVA: p<0.05, **p<0.01, ***p<0.001.

D) Expression of WNK1 or PRRC2C shRNA in CRC cells leads to decreased tumor growth. Left panel: Comparative analysis of tumor growth (mean tumor volumes) in xenografts derived from HCT116 and SW480 cell lines transfected with WNK1 or PRRC2C shRNA respectively and compared to cells containing a scrambled shRNA. There were 7 mice in the WNK1 group and 10 in the PRRC2C group. Data represent means ±SEM, t-test: *p<0.05. Right panel: WNK1 and/or PRRC2C mRNA RT-qPCR expression analysis at two time points (at day 0 from transfected cells before injection and at day 28 or 30 from tumor xenograft).

FIG. 2. Clinical relevance of MSI-driven coding region mutations in target genes in CRC patients.

Association of 5 negatively selected MSI-driven mutational events with RFS was calculated in the cohort of 164 MSI CRC patients with survival data available. Association of the boolean mutational index (see materials and methods section) calculated from the mutational status of the above 5 target genes in each tumor sample (status 0 =no mutation observed; status 1 =at least 1 mutation observed) with RFS is also shown. Also reported is the association with RFS of a series of 15 other frequent MSI mutations (6 positively selected and 9 background events) that we previously investigated in the same MSI CRC samples and published 17. Forest plot of relapse-free survival (RFS) hazard ratios (HRs) of independent univariate Cox analyses are shown. Diamonds represent the hazard ratios and horizontal bars represent the 95% confidence intervals. White squares indicates a p-value of less than 5% (worse prognosis) and black squares more than 5%.

FIG. 3: Validation of gene abrogation by siRNA approach and Validation of deleterious impact of 5 OM genes in RKO, KM12, FET and SW620 CRC cell lines

Gene expression (mRNA level) of OM related genes after knock-down by single siRNA was assessed 24 (A)

(B) Flow cytometry analysis of apoptosis (Annexin-V) of untreated (white) or TRAIL treated (black) MSI (RKO and KM12, left panel) and MSS (FET and SW620, right panel) CRC cell lines transfected either with single specific siRNA gene (WNK1, HMGXB4 and/or GART) or with scrambled siRNA (upper panels) t-test: *p<0.05, **p<0.01, ***p<0.001, ****p<0.0001 of indicated silencing condition compared to control.

FIG. 4: Experimental data of cell growth and cell migration analysis

Real-time monitoring of cell growth and cell migration using the xCELLigence system (HCT116 CRC MSI cell line). This system allows estimating cell index in real time—the parameter based on impedance measurement and reflecting the number of cells attached to the surface of the experimental chambers. Quadruplicates of three independent experiments are shown.

Tables

TABLE 1
List of NR mutations with predicted functional impact

| | Mutation | Home made | intogen | mutsig |
|---|---|---|---|---|
| Gene Symbol | frequency | adjust p-value | q-value | q-value |
| RBMX | 0.638 | 0.0E+00 | 0.0E+00 | 6.1E−10 |
| BRAF | 0.532 | 0.0E+00 | 9.7E−01 | 2.0E−01 |
| CACNA1H | 0.426 | 0.0E+00 | 2.1E−02 | 1.0E+00 |
| RNF145 | 0.468 | 1.2E−02 | 0.0E+00 | 1.0E+00 |
| APC | 0.404 | 1.2E−02 | 0.0E+00 | 1.0E+00 |
| ANTXR2 | 0.064 | 1.7E−01 | 0.0E+00 | 3.3E−01 |
| ZFP36L2 | 0.340 | 0.0E+00 | 0.0E+00 | 3.6E−01 |
| B2M | 0.277 | 0.0E+00 | 0.0E+00 | 1.7E−04 |
| C6orf229 | 0.255 | 0.0E+00 | NA | NA |
| RYR1 | 0.553 | 0.0E+00 | 8.9E−02 | 1.0E+00 |
| TTN | 0.957 | 1.2E−02 | 5.8E−11 | 1.0E+00 |
| SSPO | 0.489 | 0.0E+00 | — | 1.0E+00 |
| SALL3 | 0.319 | 0.0E+00 | — | 1.0E+00 |
| KRAS | 0.213 | 1.2E−02 | 2.3E−10 | 8.3E−01 |
| CASP8 | 0.149 | 1.2E−02 | 2.3E−10 | 1.0E+00 |
| DCHS1 | 0.447 | 0.0E+00 | 8.0E−01 | 1.0E+00 |
| INTS1 | 0.468 | 0.0E+00 | 1.7E−02 | 1.0E+00 |
| ACVR1B | 0.170 | 1.2E−02 | 3.0E−10 | 1.0E+00 |
| PIK3CA | 0.383 | 0.0E+00 | 2.7E−02 | 1.0E+00 |
| SEC63 | 0.149 | 1.2E−02 | 4.0E−10 | 1.0E+00 |
| MSH2 | 0.191 | 1.2E−02 | 7.4E−10 | 1.0E+00 |
| GOLGA8R | 0.191 | 0.0E+00 | — | NA |
| EPRS | 0.128 | 1.4E−02 | 9.5E−10 | 1.0E+00 |
| PTPRS | 0.340 | 0.0E+00 | 1.0E+00 | 1.0E+00 |
| CDC27 | 0.213 | 1.2E−02 | 2.1E−09 | 1.0E+00 |
| KIAA1161 | 0.277 | 0.0E+00 | 1.0E+00 | 1.0E+00 |
| LDLRAP1 | 0.149 | 1.2E−02 | 7.0E−09 | 1.0E+00 |
| KRTAP4-9 | 0.255 | 0.0E+00 | — | 2.0E−01 |
| METTL8 | 0.085 | 1.2E−02 | 2.6E−08 | 1.0E+00 |
| KALRN | 0.362 | 1.2E−02 | 1.1E−07 | 1.0E+00 |
| RP1L1 | 0.340 | 0.0E+00 | — | 1.0E+00 |
| NLRC5 | 0.340 | 0.0E+00 | 7.1E−02 | 1.0E+00 |
| FTSJ1 | 0.064 | 1.6E−02 | 4.3E−07 | 1.0E+00 |
| FAT4 | 0.468 | 0.0E+00 | — | 1.0E+00 |
| PGM1 | 0.149 | 1.3E−02 | 7.3E−07 | 1.0E+00 |
| FZD10 | 0.277 | 0.0E+00 | — | 1.0E+00 |
| RPL11 | 0.064 | 1.7E−02 | 1.0E−06 | 1.0E+00 |
| PIEZO1 | 0.447 | 0.0E+00 | 5.9E−01 | NA |
| RNF111 | 0.149 | 1.2E−02 | 1.5E−06 | 1.0E+00 |
| GLI2 | 0.383 | 0.0E+00 | — | 1.0E+00 |
| RHOF | 0.064 | 5.3E−02 | 2.1E−06 | 1.0E+00 |
| CCNB2 | 0.043 | 6.0E−02 | 2.1E−06 | 1.0E+00 |
| PLXNA1 | 0.362 | 0.0E+00 | 5.3E−01 | 1.0E+00 |
| OBSCN | 0.702 | 0.0E+00 | 2.4E−06 | 1.0E+00 |
| PLXNB2 | 0.362 | 0.0E+00 | 1.4E−01 | 1.0E+00 |
| ANKLE1 | 0.277 | 0.0E+00 | — | 1.0E+00 |
| SUPT5H | 0.191 | 1.2E−02 | 2.4E−06 | 1.0E+00 |
| SLITRK5 | 0.319 | 0.0E+00 | — | 1.0E+00 |
| TCF25 | 0.340 | 0.0E+00 | 1.2E−05 | 1.0E+00 |
| KLHL34 | 0.277 | 0.0E+00 | — | 1.0E+00 |
| FBN2 | 0.277 | 1.2E−02 | 3.4E−05 | 1.0E+00 |
| KMT2D | 0.574 | 0.0E+00 | NA | NA |
| SYTL1 | 0.170 | 1.2E−02 | 7.1E−05 | 1.0E+00 |
| FBN3 | 0.404 | 0.0E+00 | — | 1.0E+00 |
| OSBPL7 | 0.191 | 1.2E−02 | 1.0E−04 | 1.0E+00 |
| RYR3 | 0.553 | 0.0E+00 | — | 1.0E+00 |
| SKA3 | 0.149 | 1.2E−02 | 1.0E−04 | 1.0E+00 |
| ZNF835 | 0.255 | 0.0E+00 | — | 1.0E+00 |
| RNF43 | 0.234 | 0.0E+00 | 1.1E−04 | 1.0E+00 |
| KMT2B | 0.404 | 0.0E+00 | NA | NA |
| POLR1B | 0.128 | 1.9E−02 | 1.2E−04 | 1.0E+00 |
| ZNF536 | 0.255 | 0.0E+00 | — | 1.0E+00 |
| NBAS | 0.277 | 1.2E−02 | 2.0E−04 | 1.0E+00 |
| ZNF497 | 0.277 | 0.0E+00 | 1.2E−01 | 1.0E+00 |
| PRKRA | 0.106 | 1.6E−02 | 2.0E−04 | 1.0E+00 |
| CBX5 | 0.085 | 1.0E−01 | 2.0E−04 | 1.0E+00 |
| PCDH17 | 0.298 | 0.0E+00 | — | 1.0E+00 |
| MYO9B | 0.383 | 0.0E+00 | 1.0E+00 | 1.0E+00 |
| TM9SF1 | 0.106 | 1.5E−02 | 2.2E−04 | 1.0E+00 |
| EPPK1 | 0.340 | 0.0E+00 | 1.0E+00 | 1.0E+00 |
| TNC | 0.234 | 1.2E−02 | 4.2E−04 | 1.0E+00 |
| PXDNL | 0.255 | 0.0E+00 | — | 1.0E+00 |
| SHMT2 | 0.064 | 1.6E−01 | 5.5E−04 | 1.0E+00 |
| SPTA1 | 0.340 | 0.0E+00 | — | 1.0E+00 |
| KIAA1147 | 0.106 | 1.7E−02 | 5.5E−04 | 1.0E+00 |
| C5AR1 | 0.191 | 0.0E+00 | 1.0E+00 | 1.0E+00 |
| ATP11B | 0.085 | 6.2E−02 | 5.5E−04 | 1.0E+00 |
| DSCAML1 | 0.404 | 0.0E+00 | — | 1.0E+00 |
| CPEB2 | 0.106 | 1.8E−02 | 8.6E−04 | 1.0E+00 |
| DNAH1 | 0.362 | 1.2E−02 | 1.1E−03 | 1.0E+00 |
| ADARB2 | 0.340 | 0.0E+00 | — | 1.0E+00 |
| MYO7B | 0.362 | 0.0E+00 | — | 1.0E+00 |
| LCP2 | 0.149 | 1.2E−02 | 1.1E−03 | 1.0E+00 |
| BMPR2 | 0.298 | 1.2E−02 | 1.4E−03 | 1.0E+00 |
| CACNA1D | 0.340 | 0.0E+00 | — | 1.0E+00 |
| ESF1 | 0.149 | 1.2E−02 | 1.4E−03 | 1.0E+00 |
| HECTD4 | 0.383 | 1.2E−02 | 1.4E−03 | NA |
| LPPR3 | 0.191 | 0.0E+00 | NA | 1.0E+00 |
| LINGO3 | 0.234 | 0.0E+00 | — | 1.0E+00 |
| PROSC | 0.085 | 1.7E−02 | 1.5E−03 | 1.0E+00 |
| DSCAM | 0.383 | 0.0E+00 | — | 1.0E+00 |
| USP13 | 0.128 | 2.0E−02 | 1.8E−03 | 1.0E+00 |
| CELSR1 | 0.340 | 0.0E+00 | 1.0E+00 | 1.0E+00 |
| PTPRA | 0.106 | 5.0E−02 | 2.4E−03 | 1.0E+00 |
| NOTCH1 | 0.404 | 0.0E+00 | 7.3E−01 | 1.0E+00 |
| SEMA6C | 0.106 | 1.4E−02 | 2.4E−03 | 1.0E+00 |
| HECW2 | 0.149 | 1.2E−02 | 2.8E−03 | 1.0E+00 |
| MACF1 | 0.489 | 1.2E−02 | 3.3E−03 | 1.0E+00 |
| DNHD1 | 0.404 | 0.0E+00 | 6.6E−01 | 1.0E+00 |
| ZNF837 | 0.191 | 0.0E+00 | 1.0E+00 | 1.0E+00 |
| DCAF11 | 0.128 | 5.0E−02 | 3.5E−03 | 1.0E+00 |
| SLC4A3 | 0.277 | 0.0E+00 | — | 1.0E+00 |
| TGIF1 | 0.064 | 4.3E−01 | 3.5E−03 | 1.0E+00 |
| FAM47C | 0.255 | 0.0E+00 | — | 1.0E+00 |
| TGFBR2 | 0.213 | 1.2E−02 | 3.5E−03 | 1.0E+00 |
| FLG | 0.426 | 0.0E+00 | — | 1.0E+00 |
| MCOLN2 | 0.106 | 1.2E−02 | 4.0E−03 | 1.0E+00 |
| TPCN1 | 0.106 | 2.3E−02 | 4.6E−03 | 1.0E+00 |
| DDHD1 | 0.298 | 0.0E+00 | 8.6E−02 | 1.0E+00 |
| UBE2E1 | 0.043 | 1.6E−01 | 4.9E−03 | 1.0E+00 |
| SPEG | 0.340 | 0.0E+00 | 8.3E−01 | 1.0E+00 |
| EBF1 | 0.234 | 1.2E−02 | 4.9E−03 | 1.0E+00 |
| THSD7B | 0.298 | 0.0E+00 | — | 1.0E+00 |
| ALDH1A3 | 0.106 | 1.4E−02 | 4.9E−03 | 1.0E+00 |
| FAT3 | 0.426 | 0.0E+00 | — | 1.0E+00 |
| COCH | 0.106 | 1.2E−02 | 4.9E−03 | 1.0E+00 |
| CSPG4 | 0.298 | 0.0E+00 | 1.0E+00 | 1.0E+00 |
| FLVCR1 | 0.064 | 2.7E−02 | 4.9E−03 | 1.0E+00 |
| SRRM2 | 0.362 | 0.0E+00 | 1.0E+00 | 1.0E+00 |
| IARS | 0.149 | 1.2E−02 | 4.9E−03 | 1.0E+00 |
| ARHGAP39 | 0.170 | 0.0E+00 | — | 1.0E+00 |
| FBXW7 | 0.340 | 0.0E+00 | 1.7E−01 | 1.0E+00 |
| TUBA3D | 0.213 | 0.0E+00 | — | 1.0E+00 |
| KIF24 | 0.106 | 1.9E−02 | 4.9E−03 | 1.0E+00 |
| HLA-DRB5 | 0.043 | 2.9E−02 | 4.9E−03 | 1.0E+00 |
| ERP44 | 0.064 | 4.7E−02 | 4.9E−03 | 1.0E+00 |
| GCC2 | 0.064 | 7.1E−02 | 4.9E−03 | 1.0E+00 |
| ZNF780B | 0.064 | 1.2E−02 | 4.9E−03 | 1.0E+00 |
| GAMT | 0.064 | 2.3E−02 | 4.9E−03 | 1.0E+00 |
| ABCB6 | 0.149 | 1.2E−02 | 5.4E−03 | 1.0E+00 |
| GLG1 | 0.149 | 1.2E−02 | 6.2E−03 | 1.0E+00 |
| SOX1 | 0.106 | 1.2E−02 | 6.3E−03 | 1.0E+00 |
| HDLBP | 0.234 | 1.2E−02 | 6.4E−03 | 1.0E+00 |
| MYH10 | 0.234 | 1.2E−02 | 7.3E−03 | 1.0E+00 |
| MYO5A | 0.255 | 1.2E−02 | 7.6E−03 | 1.0E+00 |
| ATP2C1 | 0.064 | 4.2E−01 | 8.5E−03 | 1.0E+00 |
| ZNF184 | 0.064 | 2.6E−02 | 8.5E−03 | 1.0E+00 |
| TP53 | 0.234 | 1.2E−02 | 8.8E−03 | 1.0E+00 |
| MANBA | 0.128 | 1.2E−02 | 9.6E−03 | 1.0E+00 |
| SEPT6 | 0.085 | 5.7E−02 | 9.9E−03 | 1.0E+00 |
| CCDC115 | 0.064 | 5.9E−02 | 9.9E−03 | 1.0E+00 |

TABLE 2
Most significant coding NR mutations

| Gene | Mutation | IntoGen | Home | MutSig | |
|---|---|---|---|---|---|
| Name | frequency | Q-score | Made P-value | Q-score | Associated function |
| SSPO | 0.49 | — | 0.0E+00 | 1.0E+00 | Modulation of neuronal |
| | | | | | aggregation. |
| BRAF | 0.53 | 9.7E−01 | 0.0E+00 | 2.0E−01 | Proto-oncogene serine/threonine |
| | | | | | kinase |
| RYR1 | 0.55 | 8.9E−02 | 0.0E+00 | 1.0E+00 | Calcium channel in sarcoplasmic |
| | | | | | reticulum |
| INTS1 | 0.47 | 1.7E−02 | 0.0E+00 | 1.0E+00 | Involved in the small nuclear |
| | | | | | RNAs transcription |
| PTPRS | 0.34 | 1.0E+00 | 0.0E+00 | 1.0E+00 | Regulated cell growth, cycle and |
| | | | | | oncogenic transformation |
| CACNA1H | 0.43 | 2.1E−02 | 0.0E+00 | 1.0E+00 | Voltage-sensitive calcium |
| | | | | | channels |
| APC | 0.40 | 0.0E+00 | 7.4E−03 | 1.0E+00 | Negatif regulator of Wnt |
| | | | | | signaling. Tumor suppressor |
| DCHS1 | 0.45 | 8.0E−01 | 0.0E+00 | 1.0E+00 | Calcium-dependent cell-adhesion |
| | | | | | protein |
| KALRN | 0.36 | 1.1E−07 | 1.1E−02 | 1.0E+00 | Induces lamellipodia independent |
| | | | | | of its GEF activity |
| ZFP36L2 | 0.34 | 0.0E+00 | 0.0E+00 | 3.6E−01 | Destabilized mRNA |
| NOTCH1 | 0.40 | 7.3E−01 | 0.0E+00 | 1.0E+00 | Regulate cell-fate determination |
| | | | | | (including CD4+ and CD8+ cells) |
| PIK3CA | 0.38 | 2.7E−02 | 0.0E+00 | 1.0E+00 | Ras signaling. Cell growth. |
| | | | | | Survival. proliferation motility. |
| PLXNA1 | 0.36 | 5.5E−01 | 0.0E+00 | 1.0E+00 | Plays a role in axon guidance, |
| | | | | | invasive growth and cell migration |
| PLXNB2 | 0.36 | 1.3E−03 | 0.0E+00 | 1.0E+00 | Plays a role in RHOA activation |
| | | | | | and changes of the actin |
| | | | | | cytoskeleton |
| SALL3 | 0.32 | — | 0.0E+00 | 1.0E+00 | Probable transcription factor. |
| FBXW7 | 0.34 | 1.6E−01 | 0.0E+00 | 1.0E+00 | Acts as a negative regulator of |
| | | | | | JNK signaling |
| B2M | 0.28 | 0.0E+00 | 0.0E+00 | 6.1E−10 | Antigen presentation. |
| KIAA1161 | 0.28 | 1.0E+00 | 0.0E+00 | 1.0E+00 | Na |
| C6orf229 | 0.26 | — | 0.0E+00 | 1.0E+00 | Na |
| KRTAP4-9 | 0.26 | — | 0.0E+00 | 2.0E−01 | Essential for the formation of hair |
| CASP8 | 0.15 | 2.3E−10 | 1.2E−02 | 1.0E+00 | Activation cascade of caspases |
| KRAS | 0.21 | 2.3E−10 | 7.8E−03 | 8.3E−01 | Proto-oncogene serine/threonine |
| | | | | | kinase |
| MSH2 | 0.19 | 7.4E−10 | 7.4E−03 | 1.0E+00 | Component of MMR |
| CDC27 | 0.21 | 2.1E−09 | 7.4E−03 | 1.0E+00 | Component of the anaphase |
| | | | | | promoting complex/cyclosome |
| | | | | | (APC/C) |
| GOLGA8R | 0.19 | — | 0.0E+00 | 1.0E+00 | Na |
| ACVR1B | 0.17 | 3.0E−10 | 7.8E−03 | 1.0E+00 | Activin signaling. |
| | | | | | serine/threonine kinase |
| EPRS | 0.13 | 9.5E−10 | 1.1E−02 | 1.0E+00 | Calcium-dependent cell-adhesion |
| | | | | | protein |
| LDLRAP1 | 0.15 | 7.0E−09 | 1.1E−02 | 1.0E+00 | Endocytosis of low-density |
| | | | | | lipoprotein |
| METTL8 | 0.08 | 2.6E−08 | 1.1E−02 | 1.0E+00 | Probable methyltransferase |
| ANTXR2 | 0.06 | 0.0E+00 | 7.8E−03 | 3.3E−01 | Cellular interactions |

### EXAMPLE

Material & Methods

Tumor Cohort

Tumor Cohort of MSI CRC Patients Analyzed by Whole-Exome Sequencing

Forty seven patients who underwent surgical resection for MSI CRC from the Hopital Saint Antoine (Paris, France) were selected for this study. Tumor samples and adjacent normal tissue counterparts (NT) were collected and stored frozen at −80° C. before DNA extraction. DNA was purified using the Qiamp protocol (Qiagen) as recommended by the manufacturer. Informed consent was obtained for all patients. Gene expression for 30 samples from this cohort was previously analyzed on Affymetrix U133 plus 2 chips as described (23).

Tumor Cohort of MSI CRC Patients for Survival Analysis 164 MSI CRC samples with available WGA DNA were further analyzed for association between MSI mutational events and RFS (Relapse Free Survival) (17).

Exome Data Analyses

Whole-Exome Sequencing

Exome sequencing was performed on 47 pairs of MSI CRC and paired adjacent normal mucosa. For the 47 pairs of MSI CRC and paired adjacent normal mucosa, 3 μg of genomic DNA was fragmented by sonication and purified to obtain fragments of 150 to 200 bp. The oligonucleotide adapters for sequencing were ligated to DNA fragments and purified. After purification, exonic sequences were captured by hybridizing the sequences to biotinylated exon library baits, which were then captured with streptavidin-coated magnetic beads that complex with biotin (SureSelect Human All Exon Kit v5+UTR, 75 Mb). Enrichment and elution were performed according to the manufacturer's recommendations (24) (Sureselect, Agilent) and sequenced on Illumina HiSeq 2000 as paired end 75 bp. Image analysis and base calling were performed using Illumina Real-Time Analysis Pipeline version 1.14 with default parameters. Read sequence Fastq files were generated and quality control checked following Illumina's recommendations and FastQC reports.

Overall Mutation (SNV and Indel) Calling

The exome data analysis was first performed using Illumina CASAVA 1.8.2 software which includes reads mapping and variant calling. Exome-Seq data have been deposited in the European Genome-phenome Archive, accession no. EGAS00001002477. Reads were aligned against the hg19 genome build (GRCh37) with ELANDv2, a gapped and multi-seed aligner that reduces artefactual mismatches and allows the identification of small indels (<=10 nt), which is mandatory for analyzing microsatellite instability. Casava then detects single-nucleotide variants (SNV) and insertion/deletion (indel) variants independently in the tumor and normal samples. To distinguish somatic from germline variants, the results are combined and a Fisher test for base distribution between normal and tumor DNA is computed. We then applied a previously described method (25) to generate a list of somatic variants. Quality control filtering removed variants sequenced in <10 reads, with <3 variant calls or with QPHRED of <20. Variants were considered to be of somatic origin when the frequency of variant reads was ≥10% in the tumor and <5% in the normal counterpart, with significant enrichment of variant calls in the tumor as assessed by Fisher's exact test (p<0.05). Variants were then annotated with Annovar for gene symbol, gene structure location and exonic functional impact using RefGene database (hg19 version). Common polymorphisms with a reported frequency of >1% were removed following comparison with the 1000 Genomes Project database and a proprietary database of exomes from normal tissues. Variants were functionally annotated using the annotations provided by Annovar based on the LJB* databases (include SIFT scores, PolyPhen2 HDIV scores, PolyPhen2 HVAR scores, LRT scores, MutationTaster scores, MutationAssessor score, FATHMM scores, GERP++scores, PhyloP scores and SiPhy scores). When the annotation was not provided, PolyPhen-2, Sift and Provean software were performed. If at least one of the methods defined the mutation as damaging, the mutation was annotated as deleterious. The mutation incidence in each tumor was evaluated by dividing the number of somatic mutations by the number of exonic bases covered by ≥10× in both the tumor and normal samples. Mutations were classified into NR and R sequence using the microsatellite list defined with MSlsensor (see below).

Mutation Calling in Microsatellite Sequences

To extensively analyze mutations at microsatellite sequence sites, we used the software MSlsensor (26) version 0.2, a program for detecting somatic microsatellite changes. First, the list of microsatellites was generated using the scan command of MSlsensor which searches for sequences of 1 to 5 bases repeated at least 5 times in the human reference genome sequence (NCBI bui1d37.1 genome fasta file). Then, using MSlsensor msi command, the mutation status for each microsatellite site (with at least 20 mapped reads) and each tumor/normal tissue pair was estimated by comparing the read length distribution between tumor and normal samples using a Chi-square test. P-values for each microsatellite were extracted from MSlsensor outputs and used to define the microsatellite mutation status (if p-value <0.05, the microsatellite was considered to be mutated). Each microsatellite was annotated for gene symbol and gene region type location (exonic, intronic, utr5, utr3) using Annovar according to RefGene database (hg19 version).

Driver Mutation Selection in Non-Repetitive Sequences

Casava mutation (snv and indel) calling results in coding regions was defined by Anovar and outside repetitive sequences. We applied MutSigCV (v1.4) and Intogen on-line software with default parameter. Many genes previously published as potential drivers in MSI CRC were not found with those two gold-standard methods. To allow recovering these genes, we implemented a simpler method: intra-sample binomial laws are fitted to report the different probabilities of mutation from one sample to another; given a sequence, its probability to be mutated at least once is calculated in each sample according to the corresponding binomial law; these probabilities are then combined across samples using Fisher's combined probability test; the resulting statistics is then compared to an empirical null distribution drawn using intronic and synonymous mutations.

Driver Mutation Selection in Repetitive Sequences

To model the somatic mutation events, we used a modified version of the classical beta-binomial model (27). We had a shift parameter for each repeat and a lasso penalty in the same fashion as Tibshirani et al. (28) to robustly estimate the main model parameters. The regularization parameter in the lasso was chosen via a heuristic technique of L-curve (29). In the estimated beta-binomial model, Pearson residuals (30) were calculated for each repeat. We then applied the Benjamini-Hochberg-Yekutieli procedure (31) to detect significantly large residuals.

Functional Analysis in CRC Cell Lines & Primary Colon Tumor Samples

To analyze for the enrichment of genes belonging to specific biological processes (Gene ontology), mutated genes that were positively or negatively selected were analyzed using Database for Annotation, Visualization and Integrated Discovery (DAVID) (32) against H. sapiens database (p-value <0.05; number of genes ≥5).

CRC cell lines were purchased from the American Type Culture Collection. All cells were cultured in Dulbecco's Modified Eagle Medium (DMEM) supplemented with 10% fetal bovine serum, 1% penicillin/streptomycin in a humidified atmosphere at 37° C. supplied with 5% CO2. All cell lines were mycoplasma free. Primary tumors and normal colon tissues were obtained from patients with CRC undergoing surgery in Hospital Saint-Antoine between 2009 and 2014 and after informed patient consent was obtained and approval from the institutional review boards/ethics committees of Hospital Saint-Antoine, Paris, France. Patients with CRC (1998-2007) from six centers involved in a study of MSI status were described previously.

Mutation Analysis

Specific primers for exonic coding DNA repeats of negatively selected gene mutations were designed using AmplifiX software (V1.7). Polymerase chain reaction (PCR) amplification was performed on tumor DNA amplified by Whole Genome Amplification (WGA) technology using the Illustra GenomIphi DNA Amplification V2 kit (GE Healthcare, Velizy-Villacoublay, France). Absence of artefactual alteration of microsatellite sequences due to WGA was validated by comparing AFLP traces of several long microsatellites before and after WGA on a 3100 GA (Applied Biosystems, Foster City, Calif., USA). Fluorescent PCR products were run on an ABI PRISM 3100 Genetic Analyzer with GS400HD ROX size standard and POP-6 polymer and Gene mapper (V4.0) software was used to analyze negatively selected mutations in exonic microsatellite traces (Applied Biosystems, Foster City, Calif., USA).

Transient Gene Silencing by Cell Transfection and Treatments

A total of 1.25×10⁵ cells were cultured in a 6-well plate for 24 hours. Cells were then transfected with Silencer® Select siRNAs (two targets per gene) using Lipofectamine RNAimax according to the manufacturer's recommendations (Thermofisher Scientific). siRNA inhibition was assessed 48 hours post transfection by real time quantitative PCR (RT-qPCR) (Thermofisher Scientific probes). To induce apoptosis, 48 hours after transfection the cells were treated with TRAIL agent (Invitrogen) for 3 hours at 50 ng/ml (FET) or for 4 hours at 30 ng/ml (HCT116), 100 ng/ml (SW480, RKO, SW620) and incubated at 37° C., 5% CO2.

Analysis of Cell Apoptosis

Apoptosis was analysed by flow cytometry using an Annexin V-FITC and 7-Amino-Actinomycin D (7-AAD) staining kit (Beckman Coulter, Inc) 48 hours after transfection. Cells were detached using StemPro® Accutase® cell dissociation reagent at room temperature for 10 minutes and stained with reagents according to the manufacturer's recommendations. Each sample was evaluated by flow cytometry (Gallios, Beckman Coulter, Inc). Data were analysed using Kaluza® Flow Analysis Software (Beckman Coulter, Inc).

Real-Time Cell Proliferation and Migration Monitoring and Data Analysis

HCT116 cells were seeded at a density of 3×10⁴ cells/well into E-plate 16 (ACEA Biosciences, Inc., San Diego, Calif., USA) and monitored on the xCELLigence Real-Time Cell Analyser Dual Plate (RTCA DP) instrument (ACEA Biosciences, Inc) according to the manufacturer's instructions. Cell proliferation was assessed by electrodes in chambers and impedance differences within an electrical circuit were monitored by the RTCA system every 15 minutes for up to 50 hours. Cell migration was assessed using a CIM-plate device of the xCELLigence system. CIM-plate consists of two chambers separated by microporous membrane (pore size is 8 gm) attached to microelectrodes. In this case, the cell index calculated on the basis of impedance measurements reflects the number of cells that migrate through micropores monitored by the RTCA system every 15 minutes for up to 50 hours. These differences are converted into a cell index. Baseline cell index (CI) is determined by subtracting the CI for a cell-containing well from the CI of a well with only culture media. The experiments were performed in triplicate and repeated at least three times. CI was expressed as the mean ±standard error of the mean from at least three independent experiments. Results of different treatment groups were compared by two-way analysis of variance (ANOVA) using the Bonferroni post-hoc test. *p<0.05, * *p<0.01, * * *p<0.00.1.

Transfection with shRNAs and Xenografts

Cloning of the pEBVsiRNA vectors and establishment of silenced cells were performed as described previously (33). We used the DSIR program to design shRNA sequences to target the WNK1 and PRRC2C genes. RNAi sequences targeting the genes are available on request. Cells carrying the pBD650 plasmid that expressed a scrambled shRNA sequence were used as a control. Cells were plated 24h before transfection with Lipofectamine® 2000 Transfection Reagent (Thermo fisher Scientific) according to the manufacturer's recommendations. Twenty-four hours later, the cells were trypsinized and seeded in culture medium supplemented with hygromycin (125 μg/ml for HCT116 cell line or 250 μg/ml for SW480). After 2 weeks, gene silencing was monitored by RT-QPCR analysis. Ten million HCT116 or SW480 cells transfected with shRNA were injected subcutaneously into the flank of female nude mice (Charles River Laboratories) at 6 weeks of age. Tumor size was measured with caliper every 2 days over 30 (HCT116 cell line) or 28 (SW480 cell line) days. Mice were sacrificed when the tumors reached 1500 mm3. The mice were treated according to guidelines of the Ministère de la Recherche et de la Technologie, France. Statistical methods to predetermine sample size in mice experiments were not used and the experiments were not randomized. During experiments and outcome analysis, the animal group allocations were not blinded. All experiments were conducted according to the European Communities Council Directive (2010/63/UE) for the care and use of animals for experimental procedures and complied with the regulations of the French Ethics Committee in Animal Experiment «Charles Darwin» registered at the «Comité National de Réflexion Ethique sur l'Experimentation Animale » (Ile-de-France, Paris, no5). All procedures were approved by this committee. All experiments were supervised by Dr. Ada

Collura and all efforts were made to minimize suffering.

Immunohistochemistry Briefly, 4 gm thick sections of paraffin-embedded tissue samples were cut onto silane-treated Super Frost slides (CML, Nemours, France) and left to dry at 37° C. overnight. Tumor sections were deparaffinized in xylene and rehydrated in pure ethanol. Before immunostaining, antigen retrieval was performed by immersing sections in citrate buffer pH 6,0 (WNK1) (15 min 95° C.), washed twice in PBS for 3 min and treated with 3% H202-PBS for 15 minutes in order to inhibit endogenous peroxidases. After washed in PBS, slides were saturated for 25 min in 3% BSA PBS. Sections were then incubated for 1 hour at room temperature with antibody to WNK1 (dilution 1/100; clone ab128858, Abcam). After washing in PBS, secondary antibody (8114P, Cell signaling) was added for 30 minutes at room temperature. Slides were washed twice for 5 minutes in PBS and revealed using Novared kit (Vector, Burlingame, USA). Slides were washed twice in water for 5 minutes and counterstained with 10% Meyer's hematoxylin. After one wash in water, slides were dehydrated in 100% ethanol and in xylene for 30 seconds each. Apoptosis was quantified by counting the number of labelled cells with anti-caspase 3 antibody per 100 tumor cells in the most affected areas.

Survival Analysis

In the cohort of 164 MSI CRC patients, association between mutations and survival were assessed by multivariate Cox proportional-hazards regression analyses and adjusted by TNM stage using the R package “survival”. This was performed for 5 negatively selected target MSI mutations, e.g. WNK1, PRRC2C, HMGXB4, GART and RFC3. This was also performed for a Boolean mutational index that was calculated from the mutational status of the above 5 target genes in each tumor sample (status 0 =no mutation observed; status 1=at least 1 mutation observed). The proportional-hazards assumption was tested using the cox.zph function. RFS was used and defined as time from diagnosis to first relapse time or death from cancer cause only. The cut point for statistical significance was 0.05.

Results

Exome-wide analysis of MMR-deficient CRC: genomic instability at non-repetitive and repetitive DNA sequences

We examined whole exome sequencing (WES) data from 47 primary MMR-deficient CRC defined as having MSI according to international criteria (34, 35). The genome fraction covered by WES was 75 MB, including UTR (37%), coding exonic (56%) and intronic (7%) regions. Repetitive DNA sequences represent less than 3% of the genome fraction covered by WES (roughly 2 Mb out of 75 Mb), with 56% in intronic, 19% in coding exonic and 25% in UTR regions (Data not shown). Computational methods were used to identify somatic mutation events at both NR (non-repetitive) and R (repetitive) DNA sequences (see Methods). Investigations were restricted to mononucleotide R sequences since these are the most frequently affected by somatic mutations in MMR-deficient cells and are often located in coding regions or in noncoding UTR sequences endowed with putative functional activity (13). Repeats of at least 5 nucleotides in length were considered, in accordance with the definition of DNA microsatellite sequences. Mutations in R and NR sequences occurred in similar proportions, representing on average 60% and 40% of all somatic events, respectively (Data not shown). These mutations accumulated in parallel in MSI tumor samples (Data not shown; P=2.47×10⁻¹⁴; R=0.85). Relative to the fraction of covered genome, the mutation rate observed in R sequences was about 24-fold higher than in NR sequences. This large difference was expected since DNA polymerases produce far more errors in R than in NR sequences. No significant differences were observed between MSI CRC from Lynch syndrome and sporadic cases, or between tumors with different TNM stages (Data not shown). Most mutations in R sequences were deletions (79%), whereas in NR sequences they were non-synonymous substitutions. A much higher number of mutations was observed in this MSI colon tumor cohort compared to the overall incidence of mutation reported for all CRC (36) (Data not shown). This high rate of mutation resulted in a much higher proportion of genes with mutations in coding regions (6% vs 1%, Data not shown), potentially leading to many candidate driver gene mutations.

Mutation frequencies were also evaluated in coding, UTR or intronic regions (Data not shown). For both NR and R sequences, a significantly higher mutation frequency was observed in intronic compared to coding exonic and UTR regions of the tumor genome (Data not shown), expectedly. As expected, mutation frequencies in R sequences were highly dependent on the repeat length, regardless of whether these were in noncoding or coding regions (Data not shown). There was almost 100% probability of mutation if the microsatellite repeat length was longer than 12 bp, consistent with previous results (14, 15, 37). Mutation events were also more frequent in G/C nucleotide repeats compared to A/T repeats. These observations indicate that distinct models are needed to analyze the occurrence of mutations in R and NR sequences in MMR deficient tumors.

Modeling the Occurrence of Mutations in Coding, Non-Repetitive DNA Sequences Identifies Known and New Actors in MSI Colorectal Tumorigenesis

MSI colon tumors accumulate somatic mutations in R and NR DNA sequences at similar proportions. In NR sequences, mutation frequencies varied according to the nature of the nucleotide change (analysis of nucleotide substitutions in MSI tumors, Data not shown). In coding sequences, they mostly consisted of non-synonymous nucleotide substitutions that were probably deleterious in the majority of cases (>50%; Data not shown). Only a small number of events in NR sequences were indels (Data not shown), in line with a previous report (38).

We next aimed to identify mutational events in NR sequences that showed an abnormally high frequency in tumor DNA, i.e. positively selected mutational events. To do this, we used two sophisticated well-known existing models (Intogen and MutSigCV). We also implemented a simpler model of the recurrence of mutations in order to recover most of the MSI CRC putative drivers previously proposed in the literature. This basic model starts by fitting intra-sample binomial laws that describe the expected number of mutations in a sequence of given length. The probability of a given sequence to be mutated at least once is calculated in each sample according to the corresponding binomial law. These probabilities are then combined across samples using Fisher's combined probability test (Data not shown). The resulting statistics is then compared to an empirical null distribution drawn using intronic and synonymous mutations. Combination of the Intogen and MutSigCV models with this combined binomial model allowed us to identify the 141 most consensual driver genes of colon tumorigenesis (Table 1). The Top-30 driver mutated gene list is shown in Table 2 and includes recognized master genes in colorectal oncogenesis such as BRAF, APC, KRAS, and PIK3CA (Data not shown).

Modeling the Occurrence of Mutations in Repetitive Sequences Reveals Positively and Negatively Selected Events in the MSI Tumor Genome

We next aimed to identify somatic mutations in R sequences that underwent selection during MSI CRC tumorigenesis. To do this, we built a mathematical model for the occurrence of mutational events that incorporated previously described structural parameters (14) as well as others shown here to impact mutations, i.e. repeat length and nucleotide composition (Adenosine/Thymine vs Guanine/Cytosine) (Data not shown). Most allelic shifts were deletions and/or insertions of 1 bp, or more rarely 2 bp (data not shown). These were considered equally as mutant alleles in the genomic analysis of instability at mononucleotide repeats. A similar pattern was observed for dinucleotide repeats (data not shown). To build the model, only MSI-related events that occurred in mononucleotide R sequences were considered, since these largely predominate over others such as in dinucleotide repeats (9, 13). Beta-binomial logistic regression was chosen as it correctly modeled the observed occurrence and the observed dispersion of mutations. Repeat length was used as an input parameter for the model and two models were fitted: one for A/T composition and the other for G/C composition (Data not shown). The density of the model for A/T composition of repeats is not shown. Microsatellites that displayed within our model, abnormally high or low mutation frequency within UTRs or coding exonic regions are indicated. These outliers most likely correspond to positively or negatively selected mutational events. Data not shown). Overall, we identified 1050 and 561 outlier events displaying aberrant positive and negative selection in MSI CRC, respectively. These included 1376 mutations in UTR sequences (828 and 548 displaying positive or negative selection, respectively) and 235 mutations in coding sequences (222 and 13 displaying positive or negative selection, respectively). With the exception of these 13 frameshift mutations that affected coding DNA sequences (see below), negatively selected events were observed almost exclusively in noncoding microsatellites (UTRs). In contrast, positively selected mutations were observed in both coding and noncoding DNA repeats (Data not shown).

Validation of Exome-Wide Analysis of MSI and Refining the List of MSI Target Genes in MSI CRC

We next compared our results with those of the TCGA consortium which used MuTect2 caller in 53 MSI CRC. The mutation frequencies observed at microsatellite loci were highly similar in both cohorts (R=0.86; p<2.10⁻¹⁶) (Data not shown). Instability at 9 microsatellite loci was also investigated using PCR and RFLP in an independent cohort of 180 MSI CRC. Using this manual, gold standard method, very similar mutation frequencies were observed for these 9 coding repeat sequences in the two cohorts with the two methods, including 8 in which we validated the low mutation frequency (Data not shown).

According to published literature, the majority of known and extensively analyzed target gene mutations in MSI CRC were found here in MMR-deficient CRC. These included AXIN2, CDX2, BCL10, APAF1, CHCK1, PLH3, BLM, RAD50, WIP3, MBD4, CASP5 and AIM2 (Data not shown). However, TGFBR2, ACVR2A, BAX, MSH3, MSH6, IGF2R and several others remained in the group of genes with positively selected mutations in MSI tumors. Interestingly, this group mostly contained a small coding repeat (5 to 7 bp in length) whose mutation frequency was not high but was nevertheless subjected to strong positive selection pressures according to our model, e.g. UNCSB, PTEN, APC. Finally, our signature also contained a small number of target genes with a long coding repeat (9 or 10 bp in length) whose mutations were negatively selected in MSI tumors (Data not shown) and in which we further assessed the functional impact (see below).

Investigating the Interplay Between MSI, Changes in Gene Expression Level and Cancer-Related Pathways

We next tested the hypothesis that both positively and negatively selected outlier mutations in R sequences constitute major events in MSI tumorigenesis that result in pro- or anti-oncogenic impacts, respectively, during tumor progression. To do this we assessed gene ontology (GO) terms associated with these mutations and found several to be significantly enriched in such events (Data not shown). These outlier mutations were observed in cancer-related pathways known to play an important role in tumor development, e.g. Wnt/Wingless and RAF/RAS/MAPK signaling, or with antitumor immunity. Their positive or negative selection in MSI tumors were likely to accord with their expected positive or negative impact, respectively, on the activity of these pathways in CRC (Data not shown).

We then assessed whether these outlier mutations influenced the expression level of the corresponding target gene in MSI tumors. Several mutations in coding regions and in UTRs were associated with significantly altered gene expression when assessed at the mRNA level using transcriptome data from 30 MSI CRC samples (Data not shown). Due to nonsense-mediated mRNA decay (39), we mostly observed down-regulation of mutated transcripts from coding regions, as expected. The overall impact of outlier events in UTR tracts was mixed, with down- or up-regulation of a few target genes in MSI CRC. Based on these results, a list of outlier mutations expected to play an important role in MSI tumor development was proposed (Data not shown). In line with a pro-tumorigenic effect, positively selected outlier events may inactivate tumor suppressor functions by down-regulating mRNA expression or activate oncogene functions by up-regulating mRNA expression. Acting in opposition, the negatively selected outlier events could activate tumor suppressor functions by up-regulating mRNA expression or inactivate oncogene functions by down-regulating mRNA expression, thereby slowing down MSI tumorigenesis.

Functional Validation of the Deleterious Impact of Negatively Selected Coding Mutations on CRC Cells

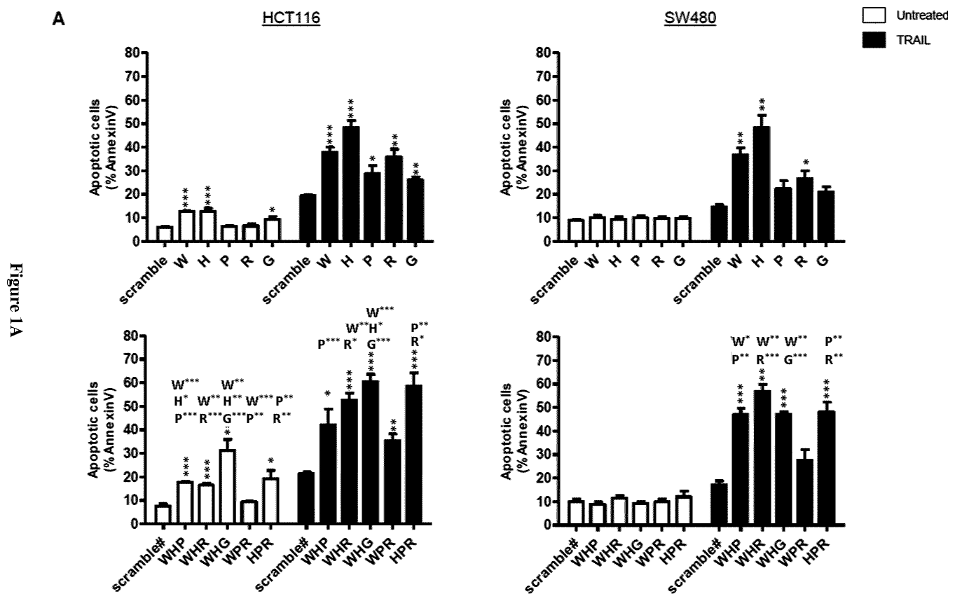

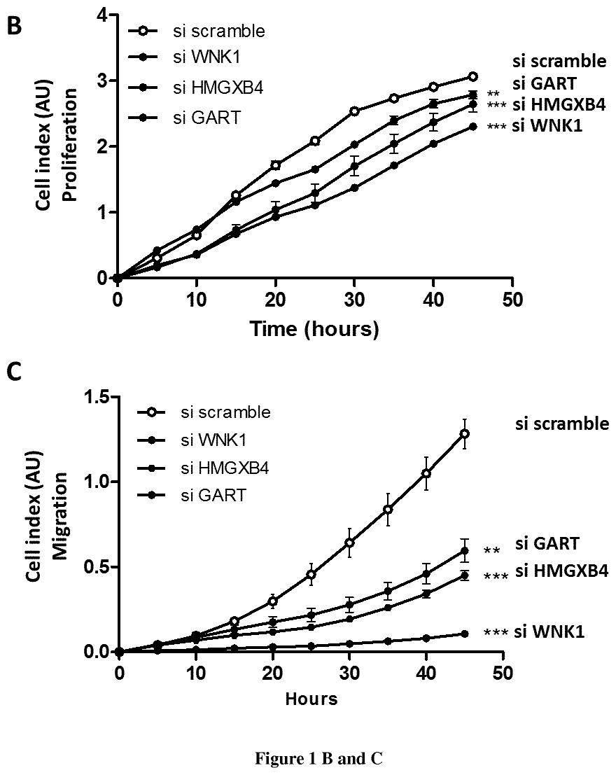

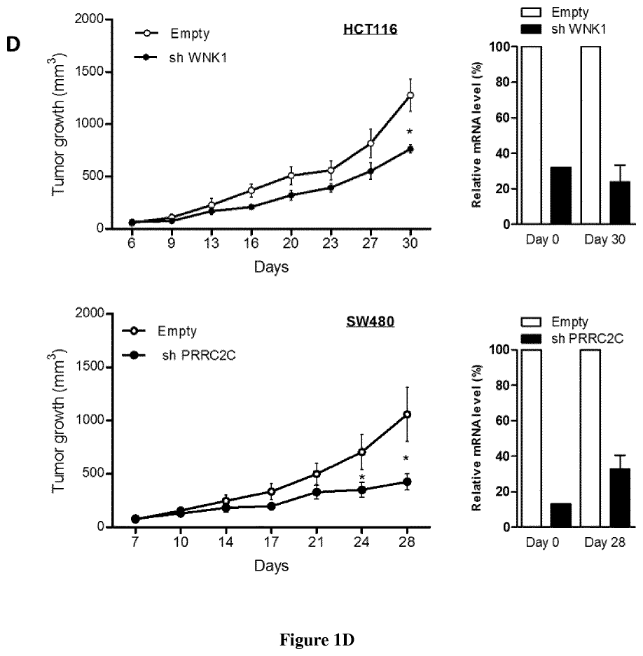

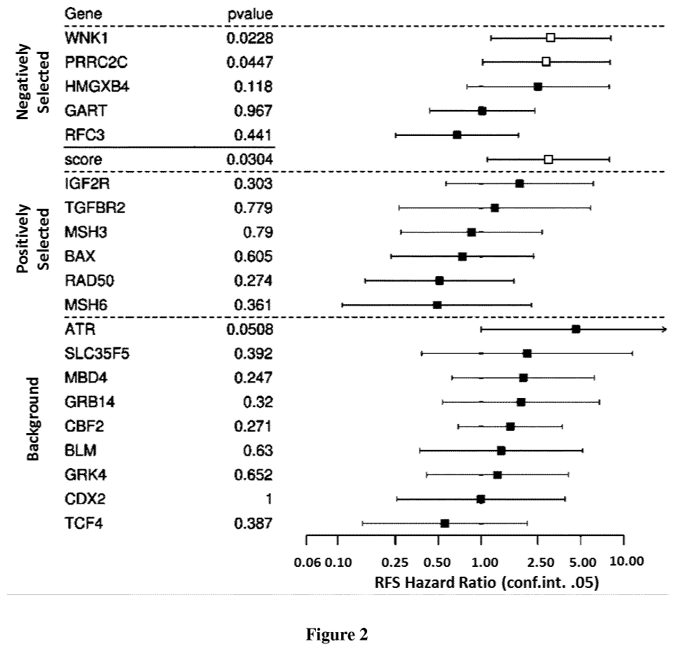

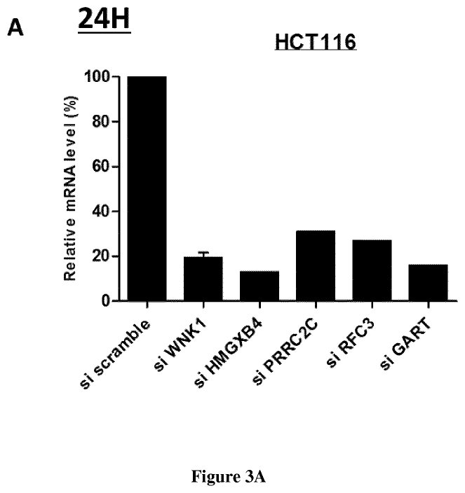

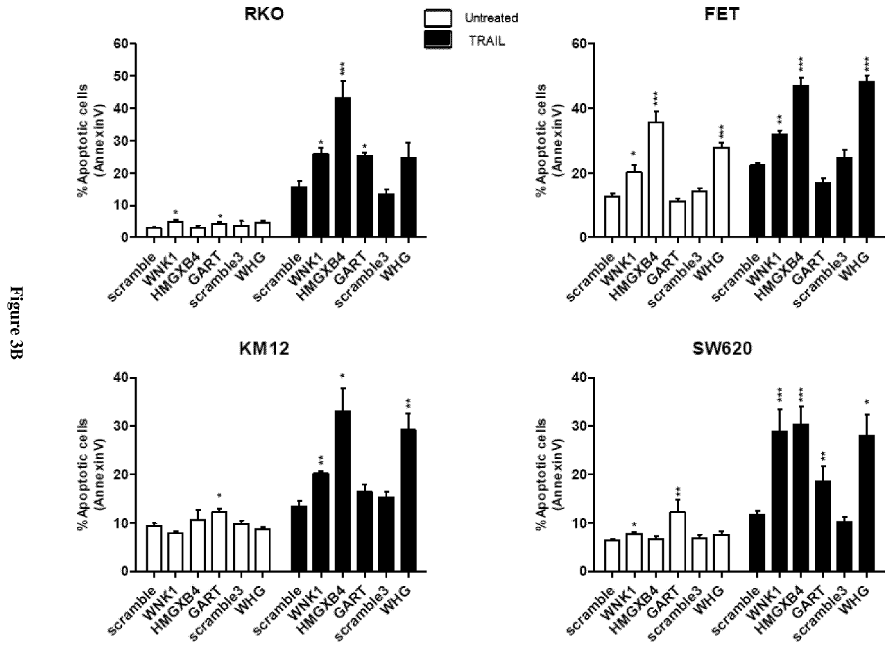

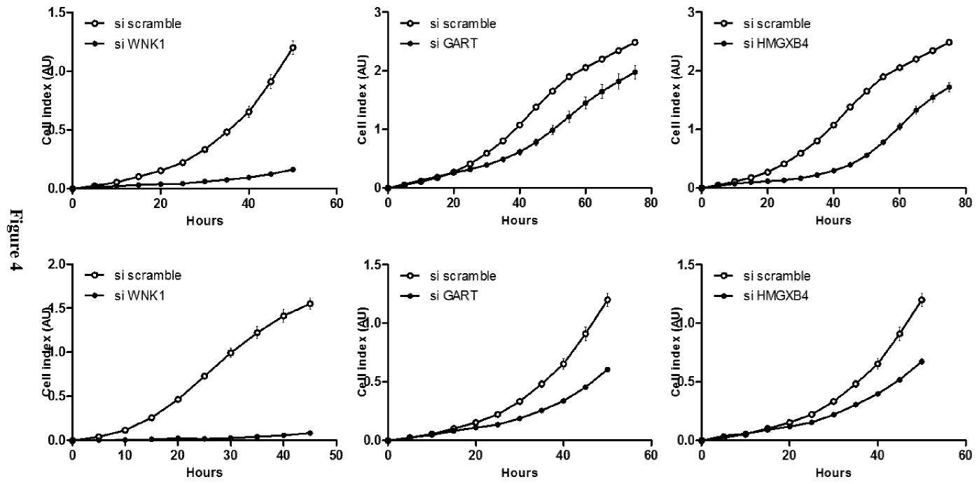

We hypothesized that negatively selected mutational events identified in the genomic screen could be deleterious for MSI tumor cells. As stated above, only a few of these events were in coding regions and led to truncation of the respective proteins (e.g. WNK1, PRRC2C, CHD2, SYCP1, GART, RXFP2, RFC3, HMGXB4) (Data not shown). Five of these target genes (WNK1, HMGXB4, PRRC2C, RFC3 and GART) were selected. To test the hypothesis that truncation of these candidate proteins due to MSI was responsible for their inactivation, we investigated the functional consequences of their silencing using siRNA and/or shRNA in CRC cell lines in vitro and in vivo using xenograft models (FIG. 1 and FIG. 3, 4). Depending on the target gene, their inactivation in CRC cells led to deleterious effects on apoptosis, proliferation and/or cell migration (FIG. 1A-C and FIG. 3, 4). Of note, the deleterious effects were greatly enhanced when several of the targets were concomitantly silenced in the same cellular models, indicating additive effects for these events in CRC cells (FIG. 1 and FIG. 3). In additional experiments, the prolonged silencing of some of these targets led to strong inhibition of tumor growth in HCT116 (MSI) and/or SW480 (MSS) xenografts (FIG. 1D and Data not shown).

Negatively Selected Events are Associated with Worse Survival of MSI CRC Patients

We next evaluated whether negatively selected coding sequence mutations that were associated with deleterious effects in CRC cells (e.g. microsatellites located in coding regions of WNK1, HMGXB4, PRRC2C, RFC3 or GART) may also be clinically relevant. An additional cohort of 164 MSI CRC patients originating from 3 clinical centers in France was analyzed by Cox survival models adjusted for TNM stage. In the overall cohort, mutated WNK1 (hazard ratio [HR], 3.1; 95% confidence interval, 1.2-8; p=0.02) and PRRC2C (hazard ratio [HR], 2.9; 95% confidence interval, 1-8.1; p=0.04) were associated with worse relapse-free survival (RFS) (FIG. 2). The HMGXB4 mutation also showed a trend for association with worse RFS (hazard ratio [HR], 2.5; 95% confidence interval, 0.78-7.8; p=0.12; FIG. 2).

To examine the overall relationship between the 5 negatively selected target gene mutations and patient survival, a mutational index value was computed to summarize this MSI target gene category. Cox modeling based on this representation was associated with significantly worse survival, suggesting an overall negative impact of these mutational events on patient outcome (hazard ratio [HR], 3; 95% confidence interval, 1.1-7.9; p=0.03; FIG. 2).

Discussion

The present work indicates that in an MMR-deficient context (9, 10, 14, 40), genomic instability generates positively selected somatic mutations in both repetitive and non-repetitive DNA sequences that are likely to contribute to the tumorigenic process. Moreover, this work also suggests that negatively selected somatic mutations arise in microsatellite repeat sequences and may have deleterious functional impacts on tumor cells. Microsatellites are widely dispersed in human genes and hence somatic mutation of these repeats is inevitable in the context of a high background of MSI. The high frequency of such mutations in MSI tumors can only be reduced by negative selection during tumorigenesis. The present results shed new light on MMR-deficient tumorigenesis and suggest that genomic instability in MSI CRC plays a dual role in achieving tumor cell transformation.

Due to the rarity of long DNA repeats in the coding genome, very few negatively selected mutational events were located in this region. Frameshift gene mutations in coding repeats are likely to result in inactivation of the corresponding truncated mutant protein, provided the mutant transcript is not degraded by NMD (11). The clearly deleterious consequences of these mutations in both MSI and MSS tumor cells is therefore of interest. We postulate that target genes for MSI can act as powerful anti-cancer drivers in MSI CRC. Their frequent somatic inactivation can impede the progress of cell transformation and lead to the regression of clones in which they occur. A major example of this was WNK1, which codes for a positive regulator of canonical Wnt/-catenin signaling and whose inactivation in different tumor types is deleterious (41, 42). A second was HMGXB4, which codes for a histone chromosomal protein belonging to the High Mobility Group (HMG)-box protein family but whose role in cancer is currently unknown. A third was GART, which codes for a phospho-ribosylglycinamide formyltransferase with a putative oncogenic role in cancer (43, 44). A fourth was RFC3, which codes for the Replication factor C subunit 3 and was previously reported to be overexpressed in several tumor types (45, 46). A fifth was PRRC2C, which codes for a proline-rich coiled-coil 2C protein whose silencing has been shown to decrease cell proliferation in lung cancer (47). The MMR-deficient tumor cells in which these mutations occurred were eliminated from the bulk of most MSI colon tumors through negative selection. However, our results also showed that the few tumors in which these mutations were detected were associated with worse patient prognosis. This suggests the anticancer impact of such mutations was counterbalanced by other oncogenic processes that remain to be identified and were responsible for the poor prognosis. Interestingly, the association with worse survival remained significant even when these mutations were considered as a single index, indicating that negatively selected MSI events could be combined in further studies aimed at evaluating their prognostic significance.

Aside from the small number of deleterious mutations found in coding sequences, negatively selected mutational events were mostly found in long, noncoding repeats located in 5′ or 3′ UTR. Although about 10% of these somatic mutations were found to alter gene expression at the RNA level, their possible functional impact requires further investigation. Interestingly, some of the negatively selected mutations identified here were found to up-regulate tumor suppressor functions during MSI tumor development, while others were observed to down-regulate oncogene functions. This is in accordance with their paradoxical activation or inactivation, respectively, during the tumorigenic process. The impact of MSI on intronic sequences was not evaluated here. This will require analysis of RNASeq data from the same tumors in order to deduce any functional consequences on gene splicing, as previously assessed for several candidate genes (16, 48).

Our analysis of the MSI colon tumor exome confirmed the majority of known target gene mutations for MSI, including those in AXIN2, CDX2, MSH6, BCL10, APAF1, WISP3, BLM, RAD50, MBD4 and CASPS. These and many other mutations in 8-10 bp repeats reported previously in the literature are thought to be key events in MSI-driven tumorigenesis (40). These mutations may have functional significance in particular contexts, for example concomitant mutations in several different genes or in the same pathway. However, we showed the frequency of most of these microsatellite mutations was not different to the background frequency expected for their length, suggesting their overall impact on tumor development may be limited. In contrast, we identified several mutations in smaller coding and noncoding DNA repeats of 5 to 7 bp in length that showed a high positive selection in MSI CRC, suggesting they confer strong advantages during tumor progression. We postulate these new candidate genes for MSI tumor progression which contain relatively short repeats represent important oncogenic driver events in MMR deficient CRC and may be more relevant for tumorigenesis than many of the MSI-related mutations reported in the past. The pathophysiological relevance and opposing functional effects of such MSI-driven events should allow major advances in the understanding of MSI tumorigenesis and in the development of personalized treatments for patients with MMR-deficient tumors.

### REFERENCES

Throughout this application, various references describe the state of the art to which this invention pertains. The disclosures of these references are hereby incorporated by reference into the present disclosure.

1. Hanahan D, Weinberg R A. Hallmarks of cancer: the next generation. Cell 2011;144:646-74.

2. Greaves M, Maley C C. Clonal evolution in cancer. Nature 2012;481:306-13.

3. Martincorena I, Raine K M, Gerstung M, et al. Universal Patterns of Selection in Cancer and Somatic Tissues. Cell 2017; 171:1029-1041 e21.

4. Bakhoum S F, Landau D A. Cancer Evolution: No Room for Negative Selection. Cell 2017; 171:987-989.

5. Leach F S, Nicolaides N C, Papadopoulos N, et al. Mutations of a mutS homolog in hereditary nonpolyposis colorectal cancer. Cell 1993; 75:1215-25.

6. Thibodeau S N, Bren G, Schaid D. Microsatellite instability in cancer of the proximal colon. Science 1993; 260:816-9.

7. Ionov Y, Peinado M A, Malkhosyan S, et al. Ubiquitous somatic mutations in simple repeated sequences reveal a new mechanism for colonic carcinogenesis. Nature 1993; 363:558-61.

8. Fishel R, Lescoe M K, Rao M R, et al. The human mutator gene homolog MSH2 and its association with hereditary nonpolyposis colon cancer. Cell 1993; 75:1027-38.

9. Cortes-Ciriano I, Lee S, Park W Y, et al. A molecular portrait of microsatellite instability across multiple cancers. Nat Commun 2017; 8:15180.

10. Hause R J, Pritchard C C, Shendure J, et al. Classification and characterization of microsatellite instability across 18 cancer types. Nat Med 2016; 22:1342-1350.

11. Duval A, Hamelin R. Mutations at coding repeat sequences in mismatch repair-deficient human cancers: toward a new concept of target genes for instability. Cancer Res 2002; 62:2447-54.

12. Cancer Genome Atlas N. Comprehensive molecular characterization of human colon and rectal cancer. Nature 2012; 487:330-7.

13. Kim T M, Laird P W, Park P J. The landscape of microsatellite instability in colorectal and endometrial cancer genomes. Cell 2013; 155:858-68.

14. Duval A, Reperant M, Compoint A, et al. Target gene mutation profile differs between gastrointestinal and endometrial tumors with mismatch repair deficiency. Cancer Res 2002; 62:1609-12.

15. Woerner S M, Yuan Y P, Benner A, et al. SelTarbase, a database of human mononucleotide-microsatellite mutations and their potential impact to tumorigenesis and immunology. Nucleic Acids Res 2010; 38:D682-9.

16. Dorard C, de Thonel A, Collura A, et al. Expression of a mutant HSP110 sensitizes colorectal cancer cells to chemotherapy and improves disease prognosis. Nat Med 2011; 17:1283-89.

17. Collura A, Lagrange A, Svrcek M, et al. Patients with colorectal tumors with microsatellite instability and large deletions in HSP110 T17 have improved response to 5-fluorouracil-based chemotherapy. Gastroenterology 2014; 146:401-11 e1.

18. Berthenet K, Boudesco C, Collura A, et al. Extracellular HSP110 skews macrophage polarization in colorectal cancer. Oncoimmunology 2016; 5:e1170264.

19. Berthenet K, Bokhari A, Lagrange A, et al. HSP110 promotes colorectal cancer growth through STAT3 activation. Oncogene 2017; 36:2328-2336.

20. Sagher D, Hsu A, Strauss B. Stabilization of the intermediate in frameshift mutation. Mutat Res 1999; 423:73-7.

21. Yamamoto H, Imai K. Microsatellite instability: an update. Arch Toxicol 2015; 89:899-921.

22. Kondelin J, Gylfe A E, Lundgren S, et al. Comprehensive evaluation of protein coding mononucleotide microsatellites in microsatellite-unstable colorectal cancer. Cancer Res 2017.

23. Marisa L, de Reynies A, Duval A, et al. Gene expression classification of colon cancer into molecular subtypes: characterization, validation, and prognostic value. PLoS Med 2013; 10:e1001453.

24. Gnirke A, Melnikov A, Maguire J, et al. Solution hybrid selection with ultra-long oligonucleotides for massively parallel targeted sequencing. Nat Biotechnol 2009; 27:182-9.

25. Assie G, Letouze E, Fassnacht M, et al. Integrated genomic characterization of adrenocortical carcinoma. Nat Genet 2014; 46:607-12.

26. Niu B, Ye K, Zhang Q, et al. MSlsensor: microsatellite instability detection using paired tumor-normal sequence data. Bioinformatics 2014; 30:1015-6.

27. Williams D A. Extra binomial variation in logistic linear models. Appl. Statist. 1982; 31:144-48.

28. Tibshirani J, Manning C D. Robust logistic regression using shift parameters. ACL 2014; 2:124-9.

29. Hansen P C, O-Leary D P. The Use of the L-Curve in the Regularization of Discrete Ill-Posed Problems SIAM J. Sci. Comput. 1993; 14:1487-1503.

30. McCullagh P, Nelder JA. Generalized linear models. CRC press 1989; 37.

31. Benjamini Y, Yekutieli D. The control of the false discovery rate in multiple testing under dependency. Ann. Statis. 2001.

32. Huang da W, Sherman B T, Lempicki R A. Bioinformatics enrichment tools: paths toward the comprehensive functional analysis of large gene lists. Nucleic Acids Res 2009; 37:1-13.

33. Biard D S, Despras E, Sarasin A, et al. Development of new EBV-based vectors for stable expression of small interfering RNA to mimick human syndromes: application to NER gene silencing. Mol Cancer Res 2005; 3:519-29.

34. Boland C R, Thibodeau S N, Hamilton S R, et al. A National Cancer Institute Workshop on Microsatellite Instability for cancer detection and familial predisposition: development of international criteria for the determination of microsatellite instability in colorectal cancer. Cancer Res 1998; 58:5248-57.

35. Buhard O, Cattaneo F, Wong Y F, et al. Multipopulation analysis of polymorphisms in five mononucleotide repeats used to determine the microsatellite instability status of human tumors. J Clin Onco12006; 24:241-51.

36. Alexandrov L B, Nik-Zainal S, Wedge D C, et al. Signatures of mutational processes in human cancer. Nature 2013; 500:415-21.

37. Duval A, Rolland S, Compoint A, et al. Evolution of instability at coding and non-coding repeat sequences in human MSI-H colorectal cancers. Hum Mol Genet 2001; 10:513-8.

38. Vogelstein B, Papadopoulos N, Velculescu V E, et al. Cancer genome landscapes. Science 2013; 339:1546-58.

39. El-Bchiri J, Buhard O, Penard-Lacronique V, et al. Differential nonsense mediated decay of mutated mRNAs in mismatch repair deficient colorectal cancers. Hum Mol Genet 2005; 14:2435-42.

40. Hamelin R, Chalastanis A, Colas C, et al. [Clinical and molecular consequences of microsatellite instability in human cancers]. Bull Cancer 2008; 95:121-32.

41. Rodan A R, Jenny A. WNK Kinases in Development and Disease. Curr Top Dev Biol 2017; 123:1-47.

42. Serysheva E, Mlodzik M, Jenny A. WNKs in Wnt/beta-catenin signaling. Cell Cycle 2014; 13:173-4.

43. Cong X, Lu C, Huang X, et al. Increased expression of glycinamide ribonucleotide transformylase is associated with a poor prognosis in hepatocellular carcinoma, and it promotes liver cancer cell proliferation. Hum Patho12014; 45:1370-8.

44. Liu X, Ding Z, Liu Y, et al. Glycinamide ribonucleotide formyl transferase is frequently overexpressed in glioma and critically regulates the proliferation of glioma cells. Pathol Res Pract 2014; 210:256-63.

45. He Z Y, Wu S G, Peng F, et al. Up-Regulation of RFC3 Promotes Triple Negative Breast Cancer Metastasis and is Associated With Poor Prognosis Via EMT. Transl Oncol 2017; 10:1-9.

46. Shen H, Cai M, Zhao S, et al. Overexpression of RFC3 is correlated with ovarian tumor development and poor prognosis. Tumour Bio12014; 35:10259-66.

47. de Miguel F J, Sharma R D, Pajares M J, et al. Identification of alternative splicing events regulated by the oncogenic factor SRSF1 in lung cancer. Cancer Res 2014; 74:1105-15.

48. Giannini G, Rinaldi C, Ristori E, et al. Mutations of an intronic repeat induce impaired MREll expression in primary human cancer with microsatellite instability. Oncogene 2004; 23 :2640-7.

1. (canceled)

2. (canceled)

3. (canceled)

4. (canceled)

5. (canceled)

6. (canceled)

7. A method for predicting the survival time of a subject suffering from a cancer comprising i) detecting at least one mutation of WNK1, HMGXB4, GART, RFC3 and/or PRRC2C in a biological sample obtained from the subject, and ii) concluding that the subject will have a short survival time when detecting said at least one mutation of WNK1, HMGXB4, GART, RFC3 and/or PRRC2C.

8. The method of claim 7, wherein the cancer is microsatellite stable cancer or microsatellite unstable cancer.

9. The method of claim 7, wherein the cancer is microsatellite stable colorectal cancer.

10. The method of claim 7, wherein the cancer is microsatellite unstable colorectal cancer.

11. (canceled)

12. A method of treating a cancer in a patient in need thereof, comprising:
i) detecting, in a biological sample obtained from the subject, one or more mutations in at least one gene selected from the group consisting of WNK1, HMGXB4, GART, RFC3 and PRRC2C, and
ii) administering at least one anti-cancer compound to the patient if the one or more mutations is detected.

13. The method of claim 12, wherein the cancer is microsatellite stable cancer or microsatellite unstable cancer.

14. The method of claim 12, wherein the cancer is microsatellite stable colorectal cancer.

15. The method of claim 12, wherein the cancer is microsatellite unstable colorectal cancer.

16. The method of claim 12, wherein the at least one anti-cancer compound is an inhibitor selected from the group consisting of: a WNK1 inhibitor, a HMGXB4 inhibitor, a GART inhibitor, a RFC3 inhibitor or a PRRC2C inhibitor.

17. The method of claim 16, wherein the inhibitor is selected from the group consisting of a small organic molecule, a polypeptide, an aptamer, an antibody, an oligonucleotide and a ribozyme.

18. The method of claim 16, wherein the inhibitor is siRNA or shRNA.

19. The method of claim 16, wherein when the one or more mutations is in the WNK1 gene, the inhibitor is a WNK1 inhibitor;
when the one or more mutations is in the HMGXB4 gene, the inhibitor is a HMGXB4 inhibitor;
when the one or more mutations is in the GART gene, the inhibitor is a GART inhibitor;
when the one or more mutations is in the RFC3 gene, the inhibitor is a RFC3 inhibitor; and
when the at least one mutation is in the PRRC2C gene, the inhibitor is a PRRC2C inhibitor.

20. The method of claim 12, wherein if the one or more mutations is detected, the patient has a short survival time.
